(19) United States
(12) Patent Application Publication
(10) Pub. No.: US 2024/0100136 A1
(43) Pub. Date: Mar. 28, 2024

(54) CHIMERIC ADAPTOR PROTEINS AND METHODS OF REGULATING GENE EXPRESSION

(71) Applicant: REFUGE BIOTECHNOLOGIES, INC., Menlo Park, CA (US)

(72) Inventors: Yueting ZHENG, Fremont, CA (US); Zhifen YANG, Menlo Park, CA (US)

(21) Appl. No.: 18/135,945

(22) Filed: Apr. 18, 2023

Related U.S. Application Data
(63) Continuation of application No. 17/902,255, filed on Sep. 2, 2022, now abandoned.
Application No. 17/902,255 is a continuation of application No. PCT/US21/20874, filed on Mar. 4, 2021.
(60) Provisional application No. 62/985,876, filed on Mar. 5, 2020.

(51) Int. Cl. A61K 39/00; A61P 37/02; C07K 14/725; C12N 15/86

(52) U.S. Cl. CPC A61K 39/0011; A61P 37/02; C07K 14/7051; C12N 15/86; A61K 2039/5156; A61K 2039/5158

(57) ABSTRACT

The present disclosure provides a method of regulating expression of a target polynucleotide in a cell. The method may comprise expressing a system in the cell, wherein the cell comprises a receptor having a ligand binding domain specific for a ligand. The method may comprise contacting the cell with the ligand that binds specifically the ligand binding domain. The system expressed in the cell may comprise a first chimeric polypeptide and a second chimeric polypeptide that are activatable upon the contacting. One of the first and second chimeric polypeptides may comprise a gene modulating polypeptide (GMP) comprising an actuator moiety linked to a cleavage recognition site. The actuator moiety may be capable of regulating the expression of the target polynucleotide in the cell. The other of the first and second chimeric polypeptides may comprise a cleavage moiety capable of cleaving the cleavage recognition site of the GMP.

### CROSS-REFERENCE

This application is a continuation of U.S. patent application Ser. No. 17/902,255, filed Sep. 2, 2022, which is a continuation of International Patent Application No. PCT/US21/20874, filed Mar. 4, 2021, which claims the benefit of U.S. Patent Application No. 62/985,876, filed on Mar. 5, 2020, which is incorporated herein by reference in its entirety.

### BACKGROUND

Regulation of cell activities can involve the binding of a ligand to a membrane-bound receptor comprising a ligand binding domain and a signaling domain. Formation of a complex between a ligand and the ligand binding domain can result in a conformational and/or chemical modification in the receptor which can result in a signal transduced within the cell. In some situations, a portion of the receptor of the signaling domain or adjacent to the signaling domain is phosphorylated (e.g., trans- and/or auto-phosphorylated), resulting in a change in its activity. These events can be coupled with secondary messengers and/or the recruitment of co-factor moieties (e.g., proteins). In some instances, the change in such portion of the receptor results in binding to other signaling moieties (e.g., co-factor proteins and/or other receptors). These other signaling moieties can be activated and then carry out various functions within a cell.

### SUMMARY

In an aspect, the present disclosure provides a method of regulating expression or activity of a target polynucleotide in a cell, comprising: (a) expressing a system in the cell, wherein the cell comprises a receptor having a ligand binding domain specific for a ligand; and (b) contacting the cell with the ligand that binds specifically the ligand binding domain, wherein the system expressed in the cell comprises: a first chimeric polypeptide and a second chimeric polypeptide that are activatable upon the contacting step (b), wherein one of the first and second chimeric polypeptides comprises a gene modulating polypeptide (GMP) comprising an actuator moiety linked to a cleavage recognition site, which actuator moiety is capable of regulating the expression or activity of the target polynucleotide in the cell, and wherein the other of the first and second chimeric polypeptides comprises a cleavage moiety capable of cleaving the cleavage recognition site of the GMP, wherein, upon the contacting of the cell by the ligand that binds specifically the ligand binding domain of the receptor, the first and second chimeric polypeptides are activated such that the cleavage moiety cleaves the cleavage recognition site and releases the actuator moiety from the GMP, thereby regulating the expression or activity of the target polynucleotide in the cell, and wherein the receptor is an endogenous receptor of the cell.

In some embodiments, the first chimeric polypeptide comprises a first adaptor moiety that is activatable to bind a first intracellular domain of the endogenous receptor. In some embodiments of any one of the method disclosed herein, the second chimeric polypeptide comprises a second adaptor moiety that is activatable to bind (i) a second intracellular domain of the endogenous receptor, (ii) the first adaptor moiety, or (ii) a downstream signaling moiety of the endogenous receptor.

In some embodiments of any one of the method disclosed herein, the first chimeric polypeptide comprises a first adaptor moiety that is activatable to bind a first downstream signaling moiety of the endogenous receptor. In some embodiments of any one of the method disclosed herein, the second chimeric polypeptide comprises a second adaptor moiety that is activatable to bind (i) the first adaptor moiety, (ii) the first downstream signaling moiety, or (iii) a second downstream signaling moiety of the endogenous receptor.

In another aspect, the present disclosure provides a method of regulating expression or activity of a target polynucleotide in a cell, comprising: (a) expressing a system in the cell, wherein the cell comprises a receptor having a ligand binding domain specific for a ligand; and (b) contacting the cell with the ligand that binds specifically the ligand binding domain, wherein the system expressed in the cell comprises: a first chimeric polypeptide and a second chimeric polypeptide that are activatable upon the contacting step (b), wherein one of the first and second chimeric polypeptides comprises a gene modulating polypeptide (GMP) comprising an actuator moiety linked to a cleavage recognition site, which actuator moiety is capable of regulating the expression or activity of the target polynucleotide in the cell, and wherein the other of the first and second chimeric polypeptides comprises a cleavage moiety capable of cleaving the cleavage recognition site of the GMP, wherein, upon the contacting of the cell by the ligand that binds specifically the ligand binding domain of the receptor, the first and second chimeric polypeptides are activated such that the cleavage moiety cleaves the cleavage recognition site and releases the actuator moiety from the GMP, thereby regulating the expression or activity of the target polynucleotide in the cell, and wherein the first chimeric polypeptide or the second chimeric polypeptide is not capable of directly binding the receptor.

In some embodiments, the first chimeric polypeptide comprises a first adaptor moiety that is activatable to bind an intracellular domain of the receptor, and wherein the second chimeric polypeptide is not capable of directly binding the receptor. In some embodiments of any one of the method disclosed herein, the second chimeric polypeptide comprises a second adaptor moiety that is activatable to bind (i) the first adaptor moiety or (ii) a downstream signaling moiety of the receptor that is activatable to bind the first adaptor moiety.

In some embodiments of any one of the method disclosed herein, the first chimeric polypeptide and the second chimeric polypeptide are not capable of directly binding the receptor. In some embodiments of any one of the method disclosed herein, the first chimeric polypeptide comprises a first adaptor moiety that is activatable to bind a downstream signaling moiety of the receptor, and wherein the second chimeric polypeptide comprises a second adaptor moiety that is activatable to bind (i) the first adaptor moiety, (ii) the downstream signaling moiety, or (iii) a different downstream signaling moiety of the receptor.

In some embodiments of any one of the method disclosed herein, the receptor is an endogenous receptor.

In some embodiments of any one of the method disclosed herein, the receptor is a heterologous receptor. In some embodiments of any one of the method disclosed herein, the heterologous receptor is a chimeric antigen receptor.

In some embodiments of any one of the method disclosed herein, the first chimeric polypeptide comprises the GMP, and wherein the second chimeric polypeptide comprises the cleavage moiety.

In some embodiments of any one of the method disclosed herein, the second chimeric polypeptide comprises the GMP, and wherein the first chimeric polypeptide comprises the cleavage moiety.

In some embodiments of any one of the method disclosed herein, the first and second chimeric polypeptides are activatable upon the contacting step (b) to form a signaling complex of the receptor.

In some embodiments of any one of the method disclosed herein, the first and second chimeric polypeptides do not bind the ligand.

In Some Embodiments of any One of the Method Disclosed Herein, the Receptor is a Transmembrane Receptor or an Intracellular Receptor.

In some embodiments of any one of the method disclosed herein, the receptor comprises at least a portion of T cell receptor (TCR). In some embodiments of any one of the method disclosed herein, the TCR comprises a co-receptor of TCR, comprising CD3, CD4, or CD8. In some embodiments of any one of the method disclosed herein, an intracellular domain of the receptor comprises at least one immunoreceptor tyrosine-based activation motif (ITAM). In some embodiments of any one of the method disclosed herein, the first adaptor moiety or the second adaptor moiety comprises LCK, FYN, ZAP-70, LAT, SLP76, ITK, PLC-γ, VAV1, NCK, GADS, GRB2, PI3K, a fragment thereof, or a combination thereof.

In some embodiments of any one of the method disclosed herein, the receptor comprises at least a portion of NKG2D. In some embodiments of any one of the method disclosed herein, the first adaptor moiety or the second adaptor moiety comprises DAP10, DAP12, PI3K, GRB2, VAV1, SYK, ZAP-70, a fragment thereof, or a combination thereof.

In some embodiments of any one of the method disclosed herein, the receptor comprises at least a portion of Toll-like receptor (TLR) selected from the group consisting of: TLR1, TLR2, TLR3, TLR4, TLR5, TLR6, TLR7, TLR8, TLR9, TLR10, TLR11, TLR12, and TLR13. In some embodiments of any one of the method disclosed herein, the first adaptor moiety or the second adaptor moiety comprises MyD88, Tube, Pelle, TIRAP, TRIF, TRAM, IRAK1, TRAK4, TRAF6, TAK1, TBK1, RIPK1, PI3K, IKK, a fragment thereof, or a combination thereof.

In another aspect, the present disclosure provides a system for regulating expression or activity of a target polynucleotide in a cell, comprising: a first chimeric polypeptide and a second chimeric polypeptide, wherein one of the first and second chimeric polypeptides comprises a gene modulating polypeptide (GMP) comprising an actuator moiety linked to a cleavage recognition site, which actuator moiety is capable of regulating the expression or activity of the target polynucleotide in the cell, and wherein the other of the first and second chimeric polypeptides comprises a cleavage moiety capable of cleaving the cleavage recognition site of the GMP, wherein the cell comprises a receptor having a ligand binding domain specific for a ligand, wherein the first and second chimeric polypeptides are activatable upon contacting of the cell by the ligand that binds specifically the ligand binding domain of the endogenous receptor, wherein, upon the contacting of the cell by the ligand, the first and second chimeric polypeptides are activated such that the cleavage moiety cleaves the cleavage recognition site and releases the actuator moiety from the GMP, thereby regulating the expression or activity of the target polynucleotide in the cell, wherein the receptor is an endogenous receptor of the cell.

In some embodiments, the first chimeric polypeptide comprises a first adaptor moiety that is activatable to bind a first intracellular domain of the endogenous receptor. In some embodiments of any one of the system disclosed herein, the second chimeric polypeptide comprises a second adaptor moiety that is activatable to bind (i) a second intracellular domain of the endogenous receptor, (ii) the first adaptor moiety, or (ii) a downstream signaling moiety of the endogenous receptor.

In some embodiments of any one of the system disclosed herein, the first chimeric polypeptide comprises a first adaptor moiety that is activatable to bind a first downstream signaling moiety of the endogenous receptor. In some embodiments of any one of the system disclosed herein, the second chimeric polypeptide comprises a second adaptor moiety that is activatable to bind (i) the first adaptor moiety, (ii) the first downstream signaling moiety, or (iii) a second downstream signaling moiety of the endogenous receptor.

In another aspect, the present disclosure provides a system for regulating expression or activity of a target polynucleotide in a cell, comprising: a first chimeric polypeptide and a second chimeric polypeptide, wherein one of the first and second chimeric polypeptides comprises a gene modulating polypeptide (GMP) comprising an actuator moiety linked to a cleavage recognition site, which actuator moiety is capable of regulating the expression or activity of the target polynucleotide in the cell, and wherein the other of the first and second chimeric polypeptides comprises a cleavage moiety capable of cleaving the cleavage recognition site of the GMP, wherein the cell comprises a receptor having a ligand binding domain specific for a ligand, wherein the first and second chimeric polypeptides are activatable upon contacting of the cell by the ligand that binds specifically the ligand binding domain of the receptor, wherein, upon the contacting of the cell by the ligand, the first and second chimeric polypeptides are activated such that the cleavage moiety cleaves the cleavage recognition site and releases the actuator moiety from the GMP, thereby regulating the expression or activity of the target polynucleotide in the cell, and wherein the first chimeric polypeptide or the second chimeric polypeptide is not capable of directly binding the receptor.

In some embodiments, the first chimeric polypeptide comprises a first adaptor moiety that is activatable to bind an intracellular domain of the receptor, and wherein the second chimeric polypeptide is not capable of directly binding the receptor. In some embodiments of any one of the system disclosed herein, the second chimeric polypeptide comprises a second adaptor moiety that is activatable to bind (i) the first adaptor moiety or (ii) a downstream signaling moiety of the receptor that is activatable to bind the first adaptor moiety.

In some embodiments of any one of the system disclosed herein, the first chimeric polypeptide and the second chimeric polypeptide are not capable of directly binding the receptor. In some embodiments of any one of the system disclosed herein, the first chimeric polypeptide comprises a first adaptor moiety that is activatable to bind a downstream signaling moiety of the receptor, and wherein the second chimeric polypeptide comprises a second adaptor moiety that is activatable to bind (i) the first adaptor moiety, (ii) the downstream signaling moiety, or (iii) a different downstream signaling moiety of the receptor.

In some embodiments of any one of the system disclosed herein, the receptor is an endogenous receptor.

In some embodiments of any one of the system disclosed herein, the receptor is a heterologous receptor. In some embodiments of any one of the system disclosed herein, the heterologous receptor is a chimeric antigen receptor.

In some embodiments of any one of the system disclosed herein, the first chimeric polypeptide comprises the GMP, and wherein the second chimeric polypeptide comprises the cleavage moiety.

In some embodiments of any one of the system disclosed herein, the second chimeric polypeptide comprises the GMP, and wherein the first chimeric polypeptide comprises the cleavage moiety.

In some embodiments of any one of the system disclosed herein, the first and second chimeric polypeptides are activatable upon the contacting to form a signaling complex of the receptor.

In some embodiments of any one of the system disclosed herein, the first and second chimeric polypeptides do not bind the ligand.

In some embodiments of any one of the system disclosed herein, the receptor is a transmembrane receptor or an intracellular receptor.

In some embodiments of any one of the system disclosed herein, the receptor comprises at least a portion of T cell receptor (TCR). In some embodiments of any one of the system disclosed herein, the TCR comprises a co-receptor of TCR, comprising CD3, CD4, or CD8. In some embodiments of any one of the system disclosed herein, an intracellular domain the receptor comprises at least one immunoreceptor tyrosine-based activation motif (ITAM). In some embodiments of any one of the system disclosed herein, the first adaptor moiety or the second adaptor moiety comprises LCK, FYN, ZAP-70, LAT, SLP76, ITK, PLC-γ, VAV1, NCK, GADS, GRB2, PI3K, a fragment thereof, or a combination thereof.

In some embodiments of any one of the system disclosed herein, the receptor comprises at least a portion of NKG2D. In some embodiments of any one of the system disclosed herein, the first adaptor moiety or the second adaptor moiety comprises DAP10, DAP12, PI3K, GRB2, VAV1, SYK, ZAP-70, a fragment thereof, or a combination thereof.

In some embodiments of any one of the system disclosed herein, the receptor comprises at least a portion of Toll-like receptor (TLR) selected from the group consisting of: TLR1, TLR2, TLR3, TLR4, TLR5, TLR6, TLR7, TLR8, TLR9, TLR10, TLR11, TLR12, and TLR13. In some embodiments of any one of the system disclosed herein, the first adaptor moiety or the second adaptor moiety comprises MyD88, Tube, Pelle, TIRAP, TRIF, TRAM, IRAK1, TRAK4, TRAF6, TAK1, TBK1, RIPK1, PI3K, IKK, a fragment thereof, or a combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1B schematically illustrate a formation of a signaling complex of a receptor or a receptor complex by a first chimeric polypeptide and a second chimeric polypeptide;

FIGS. 2A-2F schematically illustrate various configurations of a signaling complex of a receptor or a receptor complex by a first chimeric polypeptide and a second chimeric polypeptide;

FIGS. 3A-3C schematically illustrate different embodiments of an endogenous receptor that recruits a first chimeric polypeptide and a second chimeric polypeptide;

FIG. 4 schematically illustrates different expression cassettes encoding the first chimeric polypeptide or the second chimeric polypeptide;

FIG. 5A shows a proportion of T cells expressing the first chimeric polypeptide and/or the second chimeric polypeptide, and FIG. 5B shows a proportion of T cells expressing PD1 without any TCR activation following expression or activity of the first chimeric polypeptide and/or the second chimeric polypeptide;

FIG. 6 shows a proportion of T cells expressing PD1 upon TCR activation following expression or activity of the first chimeric polypeptide and/or the second chimeric polypeptide; and

FIGS. 7A and 7B show a proportion of T cells expressing PD1 upon TCR activation following expression or activity of the first chimeric polypeptide and/or the second chimeric polypeptide.

### DETAILED DESCRIPTION

It is recognized in the present disclosure that conditional gene expression systems allow for conditional regulation of one or more target genes. It is recognized in the present disclosure that conditional gene expression systems such as drug-inducible gene expression systems allow for the activation and/or deactivation of gene expression in response to a stimulus, such as the presence of a drug. It is recognized in the present disclosure that currently available systems, however, can be limited due to imprecise control, insufficient levels of induction (e.g., activation and/or deactivation of gene expression), and lack of specificity.

In view of the foregoing, there exists a considerable need for alternative methods and systems to conditionally control expression or activity of a gene in a cell.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### Definitions

As used in the specification and claims, the singular forms “a,” “an,” and “the” include plural references unless the context clearly dictates otherwise. For example, the term “a transmembrane receptor” can include a plurality of transmembrane receptors.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The terms “cell death” or “death of a cell,” as used interchangeably herein, can refer to a process or event that causes a cell to cease and/or diminish normal metabolism in vivo or in vitro. Cell death can be induced by the cell itself (self-induced) or by another cell (e.g., another cell of the same type or a different type). In some cases, cell death can include, but are not limited to, programmed cell death (i.e., apoptosis), gradual death of the cells as occurs in diseased states (i.e., necrosis), and more immediate cell death such as toxicity (e.g., cytotoxicity, such as acute cytotoxicity). In some cases, cell apoptosis can be extrinsic (e.g., via signaling through a cell surface receptor, such as a death receptor) or intrinsic (e.g., via mitochondrial pathway).

The term “receptor,” as used herein, refers to a molecule (e.g., a polypeptide) that has an affinity for a given ligand. Receptors can be naturally occurring or synthetic molecules. The given ligand (or ligand) can be naturally occurring or synthetic molecules. Receptors can be employed in an unaltered state or as aggregates with other species (e.g., with one or more co-receptors, one or more adaptors, lipid rafts, etc.). Examples of receptors may include, but are not limited to, cell membrane receptors, soluble receptors, cloned receptors, recombinant receptors, complex carbohydrates and glycoproteins hormone receptors, drug receptors, transmitter receptors, autacoid receptors, cytokine receptors, antibodies, antibody fragments, engineered antibodies, antibody mimics, molecular recognition units, adhesion molecules, agglutinins, integrins, selectins, nucleic acids and synthetic heteropolymers comprising amino acids, nucleotides, carbohydrates or nonbiologic monomers, including analogs and derivatives thereof, and conjugates or complexes formed by attaching or binding any of these molecules to a second molecule.

The term “cell membrane,” as used herein, refers to the boundary membrane, external membrane, interfacial membrane, protoplasmic membrane, or cell wall that separates the protoplasm of the cell from the outside. Thus, the term “cell membrane receptor” or “transmembrane receptor,” as used here, refers to a receptor in the boundary membrane, external membrane, interfacial membrane, protoplasmic membrane, or cell wall that separates the protoplasm of the cell from the outside.

The term “antigen,” as used herein, refers to a molecule or a fragment thereof (e.g., ligand) capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen.

The term “antibody,” as used herein, refers to a proteinaceous binding molecule with immunoglobulin-like functions. The term antibody includes antibodies (e.g., monoclonal and polyclonal antibodies), as well as variants thereof. Antibodies include, but are not limited to, immunoglobulins (Ig's) of different classes (i.e. IgA, IgG, IgM, IgD and IgE) and subclasses (such as IgG1, IgG2, etc.). A variant can refer to a functional derivative or fragment which retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The terms “Fc receptor” or “FcR,” as used herein, generally refers to a receptor, or any variant thereof, that can bind to the Fc region of an antibody. In certain embodiments, the FcR is one which binds an IgG antibody (a gamma receptor, Fcgamma R) and includes receptors of the Fcgamma RI(CD64), Fcgamma RII(CD32), and Fcgamma RIII(CD16) subclasses, including allelic variants and alternatively spliced forms of these receptors. Fcgamma RH receptors include Fcgamma RIIA (an “activating receptor”) and Fcgamma RIIB (an “inhibiting receptor”), which have similar amino acid sequences that differ primarily in the cytoplasmic domains thereof. The term “FcR” also includes the neonatal receptor, FcRn, which is responsible for the transfer of maternal IgGs to the fetus.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer (e.g., transgene). A non-native gene can also refer to a naturally occurring nucleic acid or polypeptide sequence that comprises mutations, insertions and/or deletions (e.g., non-native sequence).

The terms “target polynucleotide” and “target nucleic acid,” as used herein, refer to a nucleic acid or polynucleotide which is targeted by an actuator moiety of the present disclosure. A target polynucleotide can be DNA (e.g., endogenous or exogenous). DNA can refer to template to generate mRNA transcripts and/or the various regulatory regions which regulate transcription of mRNA from a DNA template. A target polynucleotide can be a portion of a larger polynucleotide, for example a chromosome or a region of a chromosome. A target polynucleotide can refer to an extrachromosomal sequence (e.g., an episomal sequence, a minicircle sequence, a mitochondrial sequence, a chloroplast sequence, etc.) or a region of an extrachromosomal sequence. A target polynucleotide can be RNA. RNA can be, for example, mRNA which can serve as template encoding for proteins. A target polynucleotide comprising RNA can include the various regulatory regions which regulate translation of protein from an mRNA template. A target polynucleotide can encode for a gene product (e.g., DNA encoding for an RNA transcript or RNA encoding for a protein product) or comprise a regulatory sequence which regulates expression or activity of a gene product. In general, the term “target sequence” refers to a nucleic acid sequence on a single strand of a target nucleic acid. The target sequence can be a portion of a gene, a regulatory sequence, genomic DNA, cell free nucleic acid including cfDNA and/or cfRNA, cDNA, a fusion gene, and RNA including mRNA, miRNA, rRNA, and others. A target polynucleotide, when targeted by an actuator moiety, can result in altered gene expression and/or activity. A target polynucleotide, when targeted by an actuator moiety, can result in an edited nucleic acid sequence. A target nucleic acid can comprise a nucleic acid sequence that may not be related to any other sequence in a nucleic acid sample by a single nucleotide substitution. A target nucleic acid can comprise a nucleic acid sequence that may not be related to any other sequence in a nucleic acid sample by a 2, 3, 4, 5, 6, 7, 8, 9, or 10 nucleotide substitutions. In some embodiments, the substitution may not occur within 5, 10, 15, 20, 25, 30, or 35 nucleotides of the 5′ end of a target nucleic acid. In some embodiments, the substitution may not occur within 5, 10, 15, 20, 25, 30, 35 nucleotides of the 3′ end of a target nucleic acid.

The terms “transfection” or “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

The term “vector,” as used herein, can refer to a nucleic acid molecule capable transferring or transporting a payload nucleic acid molecule. The payload nucleic acid molecule can be generally linked to, e.g., inserted into, the vector nucleic acid molecule. A vector may include sequences that direct autonomous replication in a cell, or may include sequences sufficient to allow integration into host cell gene (e.g., host cell DNA). Examples of a vector may include, but are not limited to, plasmids (e.g., DNA plasmids or RNA plasmids), transposons, cosmids, bacterial artificial chromosomes, and viral vectors.

A “plasmid,” as used herein, generally refers to a non-viral expression vector, e.g., a nucleic acid molecule that encodes for genes and/or regulatory elements necessary for the expression of genes. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to Gamma-retroviral, Alpha-retroviral, Foamy viral, lentiviral, adenoviral, or adeno-associated viral vectors.

A vector of any of the embodiments of the present disclosure can comprise exogenous, endogenous, or heterologous control sequences such as promoters and/or enhancers. An “endogenous” control sequence is one which is naturally linked to a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular gene therapy.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The term “regulating” with reference to expression or activity, as used herein, refers to altering the level of expression or activity. Regulation can occur at the transcriptional level, post-transcriptional level, translational level, and/or post-translational level.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “variant,” when used herein with reference to a polypeptide, refers to a polypeptide related, but not identical, to a wild type polypeptide, for example either by amino acid sequence, structure (e.g., secondary and/or tertiary), activity (e.g., enzymatic activity) and/or function. Variants include polypeptides comprising one or more amino acid variations (e.g., mutations, insertions, and deletions), truncations, modifications, or combinations thereof compared to a wild type polypeptide. Variants also include derivatives of the wild type polypeptide and fragments of the wild type polypeptide.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “gene modulating polypeptide” or “GMP,” as used herein, refers to a polypeptide comprising at least an actuator moiety capable of regulating expression or activity of a gene and/or editing a nucleic acid sequence. A GMP can comprise additional peptide sequences which are not directly involved in modulating gene expression, for example targeting sequences, polypeptide folding domains, etc.

The term “actuator moiety,” as used herein, refers to a moiety which can regulate expression or activity of a gene and/or edit a nucleic acid sequence, whether exogenous or endogenous. An actuator moiety can regulate expression of a gene at the transcriptional level, post-transcriptional level, translational level, and/or post-translation level. An actuator moiety can regulate gene expression at the transcription level, for example, by regulating the production of mRNA from DNA, such as chromosomal DNA or cDNA. In some embodiments, an actuator moiety recruits at least one transcription factor that binds to a specific DNA sequence, thereby controlling the rate of transcription of genetic information from DNA to mRNA. An actuator moiety can itself bind to DNA and regulate transcription by physical obstruction, for example preventing proteins such as RNA polymerase and other associated proteins from assembling on a DNA template. An actuator moiety can regulate expression of a gene at the translation level, for example, by regulating the production of protein from mRNA template. In some embodiments, an actuator moiety regulates gene expression at a post-transcriptional level by affecting the stability of an mRNA transcript. In some embodiments, an actuator moiety regulates gene expression at a post-translational level by altering the polypeptide modification, such as glycosylation of newly synthesized protein. In some embodiments, an actuator moiety regulates expression of a gene by editing a nucleic acid sequence (e.g., a region of a genome). In some embodiments, an actuator moiety regulates expression of a gene by editing an mRNA template. Editing a nucleic acid sequence can, in some cases, alter the underlying template for gene expression.

The actuator moiety may comprise a Cas protein or a modification thereof. A Cas protein referred to herein can be a type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon-optimized homologue of a Cas protein. A Cas protein can be enzymatically inactive, partially active, constitutively active, fully active, inducible active and/or more active, (e.g. more than the wild type homologue of the protein or polypeptide.). A Cas protein can be Cas9. A Cas protein can be Cpf1. A Cas protein can be C2c2. A Cas protein can be Cas13 (e.g., Cas13a, Cas13b, Cas13c, or Cas13d). A cas protein can be Cas12, or a functional variant thereof. A Cas protein can be Cas12e. A Cas protein (e.g., variant, mutated, enzymatically inactive and/or conditionally enzymatically inactive site-directed polypeptide) can bind to a target nucleic acid. A Cas protein (e.g., variant, mutated, enzymatically inactive and/or conditionally enzymatically inactive endoribonuclease) can bind to a target RNA or DNA.

The terms “deactivated nuclease” or “dead nuclease,” as used interchangeably herein, can refer to a nuclease, wherein the function of the nuclease is entirely or partially deactivated. In a case where the nuclease is a Cas protein, a deactivated/dead Cas nuclease may be referred to as “dCas” (e.g., dCas9).

The term “crRNA,” as used herein, can generally refer to a nucleic acid with at least about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes, S. aureus, etc.). crRNA can generally refer to a nucleic acid with at most about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes, S. aureus, etc.). crRNA can refer to a modified form of a crRNA that can comprise a nucleotide change such as a deletion, insertion, or substitution, variant, mutation, or chimera. A crRNA can be a nucleic acid having at least about 60% sequence identity to a wild type exemplary crRNA (e.g., a crRNA from S. pyogenes, S. aureus, etc.) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary crRNA sequence (e.g., a crRNA from S. pyogenes S. aureus, etc.) over a stretch of at least 6 contiguous nucleotides.

The term “tracrRNA,” as used herein, can generally refer to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc.). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc.). tracrRNA can refer to a modified form of a tracrRNA that can comprise a nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes S. aureus, etc.) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S. aureus, etc.) sequence over a stretch of at least 6 contiguous nucleotides.

As used herein, a “guide nucleic acid” can refer to a nucleic acid that can hybridize to another nucleic acid. A guide nucleic acid can be RNA. A guide nucleic acid can be DNA. The guide nucleic acid can be programmed to bind to a sequence of nucleic acid site-specifically. The nucleic acid to be targeted, or the target nucleic acid, can comprise nucleotides. The guide nucleic acid can comprise nucleotides. A portion of the target nucleic acid can be complementary to a portion of the guide nucleic acid. The strand of a double-stranded target polynucleotide that is complementary to and hybridizes with the guide nucleic acid can be called the complementary strand. The strand of the double-stranded target polynucleotide that is complementary to the complementary strand, and therefore may not be complementary to the guide nucleic acid can be called noncomplementary strand. A guide nucleic acid can comprise a polynucleotide chain and can be called a “single guide nucleic acid.” A guide nucleic acid can comprise two polynucleotide chains and can be called a “double guide nucleic acid.” If not otherwise specified, the term “guide nucleic acid” can be inclusive, referring to both single guide nucleic acids and double guide nucleic acids.

A guide nucleic acid can comprise a segment that can be referred to as a “nucleic acid-targeting segment” or a “nucleic acid-targeting sequence.” A nucleic acid-targeting segment can comprise a sub-segment that can be referred to as a “protein binding segment” or “protein binding sequence” or “Cas protein binding segment”.

The terms “cleavage recognition sequence” or “cleavage recognition site.” as used herein, with reference to peptides, refers to a site of a peptide at which a chemical bond, such as a peptide bond or disulfide bond, can be cleaved. Cleavage can be achieved by various methods. Cleavage of peptide bonds can be facilitated, for example, by an enzyme such as a protease.

The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a GMP) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. A fusion protein can be a non-native sequence in a Cas protein.

Thus, in some embodiments, an actuator moiety may comprise a fusion polypeptide. The fusion polypeptide may comprise two or more fragments that each confer at least one activity selected from the group consisting of: nuclease activity, methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, and demyristoylation activity.

In some cases, the actuator moiety may comprise a fusion polypeptide, and the fusion polypeptide may comprise two fragments that each confer (i) a nuclease activity (or modifications thereof, e.g., Cas activity or reduced Cas activity) and (ii) a hydrolase activity (e.g., cytidine deaminase activity). In some examples, the actuator moiety comprising the fusion polypeptide may be a nucleobase editor. The term “nucleobase editor” or “base editor,” as used interchangeably herein, can refer to an agent comprising a polypeptide that is capable of making a modification to a nucleobase (e.g., A, T, C, G, or U) within a nucleic acid sequence (e.g., DNA or RNA). In some cases, the base editor (e.g., deaminase) may be capable of deaminating a base within a nucleic acid. In some cases, the base editor may be capable of deaminating a base within a DNA molecule. In some cases, the base editor may be capable of deaminating a cytosine (C) in DNA. In some cases, the base editor may be capable of excising a base within a DNA molecule. In some cases, the base editor may be capable of excising an adenine, guanine, cytosine, thymine or uracil within a nucleic acid (e.g., DNA or RNA) molecule. In some cases, the base editor may be a fusion protein comprising a programmable nucleic acid binding protein (e.g., a nuclease as provided in the present disclosure, such as Cas or dCas) fused to a cytidine deaminase. In some cases, the base editor may be fused to a uracil binding protein (UBP), such as a uracil DNA glycosylase (UDG). In some cases, the base editor may be fused to a nucleic acid polymerase (NAP) domain. In some cases, the NAP domain may be a translesion DNA polymerase. In some cases, the base editor may comprise a programmable nucleic acid binding protein, a cytidine deaminase, and a UBP (e.g., UDG). In some cases, the base editor may comprise a programmable nucleic acid binding protein, a cytidine deaminase, and a nucleic acid polymerase (e.g., a translesion DNA polymerase). In some cases, the base editor comprises a programmable nucleic acid binding protein, a cytidine deaminase, a UBP (e.g., UDG), and a nucleic acid polymerase (e.g., a translesion DNA polymerase).

In some examples, the base editor may introduce one or more transition mutations (e.g., C to T, G to A, A to G, or T to C) without requiring double stranded breaks in many cell types and organisms, including mammals.

In some cases, the actuator moiety may comprise a fusion polypeptide, and the fusion polypeptide may comprise two fragments that each confer (i) a nuclease activity (or modifications thereof, e.g., Cas activity or reduced Cas activity) and (ii) a polymerase activity (e.g., DNA or RNA polymerase activity). As used here, the term “polymerase” can refer to a polypeptide that is able to catalyze addition of one or more nucleotides or analogs thereof (e.g., natural or synthetic nucleotides) to a nucleic acid molecule in a template dependent manner. In an example, an DNA insertion sequence encoded by a template RNA molecule may be added to a 3′-end of a target DNA molecule by action of a polymerase (e.g., reverse transcriptase). Examples of a polymerase may include, but are not limited to, (i) polymerases isolated from Thermus aquaticus, Thermus thermophilus, Pyrococcus woesei, Pyrococcus furiosus, Thermococcus litoralis, and Thermotoga maritima, (ii) E. coli DNA polymerase I, the Klenow fragment of E. coli DNA polymerase I, T4 DNA polymerase, T5 DNA polymerase, T7 DNA polymerase, (iii) T7, T3, SP6 RNA polymerases, and (iv) AMV, M-MLV and HIV reverse transcriptases.

In some examples, the actuator moiety may comprise a fusion polypeptide, and the fusion polypeptide may comprise (i) a Cas protein or modifications thereof (e.g., deactivated Cas or Cas nickase) that is coupled (e.g., covalently coupled) to (ii) a reverse transcriptase. The Cas protein may be configured to only nick one strand of a target nucleic acid (e.g., one strand of a double stranded DNA molecule). The reverse transcriptase may be configured to generate a new nucleic acid sequence (e.g., a new DNA polynucleotide stand) by coping from a nucleic acid template (e.g., a RNA template). Such actuator moiety may function in conjunction with an engineered gRNA (i.e. prime editing gRNA, or pegRNA). The pegRNA may comprise a plurality of segments. The plurality of segments may comprise (i) a nucleic acid-targeting segment (e.g., spacer region of a gRNA), (ii) a Cas protein-binding segment (e.g., as two separate crRNA and tracrRNA molecules, or as a single scaffold molecule), (iii) a reverse transcriptase template segment encoding a desired nucleic acid edit, and (iv) a binding segment that binds to the nicked strand of the target nucleic acid. In an example, the reverse transcriptase template segment of the pegRNA may encode a desired DNA sequence. Alternatively, the reverse transcriptase template segment of the pegRNA may encode a complimentary DNA sequence having complementarity to a desired DNA sequence, such that when the complimentary DNA sequence is introduced to a first strand of the target gene, the desired DNA sequence may be subsequently added to a second and opposite strand of the target gene (e.g., via one or more DNA repair mechanisms).

In an example, a fusion complex of (i) an actuator moiety comprising the Cas protein and the reverse transcriptase and (ii) a pegRNA may introduce one or more transition mutations (e.g., C to T, G to A, A to G, or T to C) without requiring double stranded breaks in many cell types and organisms, including mammals. Alternatively or in addition to, such fusion complex may perform one or more transversion mutations (e.g., C to A, C to G, G to C, G to T, A to C, A to T, T to A, and T to G), e.g., for T-A to A-T mutation needed to correct sickle cell disease, without requiring double stranded breaks in many cell types and organisms, including mammals. Alternatively or in addition to, such fusion complex may introduce an indel (e.g., an insertion and/or deletion) to the target nucleic acid or target gene. The fusion complex may introduce an addition of at least 1, 2, 3, 4, 5, 6, 7, 8, 9, 10, 15, 20, 25, 30, 35, 40, 45, 50, 55, 60, 65, 70, 75, 80, 85, 90, or more nucleotides to the target gene. The fusion complex may introduce an addition of at most 90, 85, 80, 75, 70, 65, 60, 55, 50, 45, 40, 35, 30, 25, 20, 15, 10, 9, 8, 7, 6, 5, 4, 3, 2, or 1 nucleotide to the target gene. The fusion complex may introduce a deletion of at least 1, 2, 3, 4, 5, 6, 7, 8, 9, 10, 15, 20, 25, 30, 35, 40, 45, 50, 55, 60, 65, 70, 75, 80, 85, 90, or more nucleotides to the target gene. The fusion complex may introduce a deletion of at most 90, 85, 80, 75, 70, 65, 60, 55, 50, 45, 40, 35, 30, 25, 20, 15, 10, 9, 8, 7, 6, 5, 4, 3, 2, or 1 nucleotide to the target gene. The fusion complex may or may not introduce a frameshift in the gene.

In some cases, an engineered gRNA (e.g., a pegRNA) may be coupled (e.g., covalently or non-covalently coupled) to a moiety (e.g., a polypeptide molecule) that confers at least one activity selected from the group consisting of: nuclease activity, methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, and demyristoylation activity. In an example, a pegRNA may be operatively coupled to a nucleic acid polymerase (e.g., a reverse transcriptase) by action of the nucleic acid polymerase recognizing and non-covalently binding to a fragment (e.g., a loop structure) of the pegRNA. In such a case, the nucleic acid polymerase may or may not be covalently coupled to a nuclease (e.g., a Cas protein or a dCas protein).

As used herein, the “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “chimeric antigen receptor” or alternatively a “CAR” may be used herein to refer to a recombinant polypeptide construct comprising at least an extracellular antigen binding domain, a transmembrane domain, and a cytoplasmic signaling domain (also referred to herein as “an intracellular or intrinsic signaling domain”) comprising a functional signaling domain derived from a stimulatory molecule. In some cases, the stimulatory molecule may be the zeta chain associated with the T cell receptor complex. In some cases, the intracellular signaling domain further comprises one or more functional signaling domains derived from at least one costimulatory molecule. In some cases, the costimulatory molecule may comprise 4-1BB (i.e., CD137), CD27, and/or CD28. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane. In some cases, the CAR may further comprise a GMP, as described in the present disclosure.

The CAR, as used herein, may be a first-, second-, third-, or fourth-generation CAR system, a functional variant thereof, or any combination thereof. First-generation CARs (e.g., CD19R or CD19CAR) include an antigen binding domain with specificity for a particular antigen (e.g., an antibody or antigen-binding fragment thereof such as an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3 ζ receptor or FcεRIγ). Second-generation CARs modify the first-generation CAR by addition of a co-stimulatory domain to the intracellular signaling domain portion of the CAR (e.g., derived from co-stimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple co-stimulatory domains to the intracellular signaling domain portion of the CAR (e.g., CD3ζ-CD28-OX40, or CD3ζ-CD28-41BB). Fourth-generation CARs modify second- or third-generation CARs by the addition of an activating cytokine (e.g., IL-12, IL-23, or IL-27) to the intracellular signaling portion of the CAR (e.g., between one or more of the costimulatory domains and the CD3ζ ITAM domain) or under the control of a CAR-induced promoter (e.g., the NFAT/IL-2 minimal promoter).

The term “conditionally enhancing expression” refers to expression of a polypeptide sequence (e.g., an endogenous polypeptide sequence, a chimeric polypeptide sequence, etc.) that occurs subject to one or more requirements rather than continually. Upon increasing, maintaining, and/or decreasing of the expression of the polypeptide sequence in a cell (e.g., an immune cell, a stem cell, etc.), the cell may be contacted with a stimulant (e.g., a ligand or an antigen) to initiate the conditional enhancement of expressing the polypeptide sequence in the cell. In some cases, the cell may not have begun expression the polypeptide sequence prior to at least a first contact with the stimulant. In some cases, the cell may have begun expression of the polypeptide sequence, and after the expression of the polypeptides sequence is plateaued out or decreased, the cell may be contacted with the stimulant to initiate the conditional enhancement of expressing the polypeptide sequence in the cell. The cell may be ex vivo (e.g., in vitro) or in vivo (e.g., administered to a subject). In some cases, the conditional enhancement of expressing the polypeptide sequence in the cell may be temporary or permanent. In some cases, the cell may be contacted with the stimulant at least about 1, 2, 3, 4, 5, 6, 7, 8, 9, 10 times, or more. In some cases, the cell may be contacted with the stimulant at most about 10, 9, 8, 7, 6, 5, 4, 3, 2, or 1 time.

In some cases, a continual expression of a polypeptide sequence (e.g., Cas, dCas, or a different protein that is endogenous or exogenous to the cell) may have an off-target effect on a host cell, e.g., cell cytotoxicity. In such a case, conditionally promoting and/or enhancing expression of the polypeptide sequence (e.g., via contacting the cell with a stimulant) may be beneficial, at least for a reason that cell cytotoxicity may be controlled (e.g., diminished or prevented). Alternatively or in addition to, conditionally promoting and/or enhancing expression of the polypeptide sequence may be beneficial in that a continual metabolic burden of the host cell to synthesize the polypeptide sequence can be controlled (e.g., diminished or prevented). Without wishing to be bound by theory, controlling the metabolic burden of the host cell can improve viability, proliferation, and/or function of the host cell.

The terms “operatively linked” and “under the operative control” may be used herein interchangeably to refer to two sequences (e.g., two nucleotide sequences, two polypeptide sequences, a nucleotide sequence and a polypeptide sequence) that are either physically linked or are functionally linked so that at least one of the sequences can act on the other sequence. In some cases, a gene regulatory sequence (e.g., a promoter) and an additional nucleotide sequence (e.g., a gene of interest, a transgene, etc.), are operatively linked if the expression (e.g., transcription and translation) of the additional nucleotide sequence can be governed by the gene regulatory sequence. Accordingly, the gene regulatory sequence and the additional nucleotide sequence to be expressed may be physically linked to each other, e.g., by inserting the gene regulatory sequence at or adjacent to a 5′ end of the additional nucleotide sequence to be expressed. Alternatively, the gene regulatory sequence and the additional nucleotide sequence to be expressed may be merely in physical proximity so that the gene regulatory sequence is functionally linked to the additional nucleotide sequence to be expressed. In some cases, the two sequences that are operatively linked may be separated by at least 5, 10, 20, 40, 60, 80, 100, 300, 500, 1500 bp, or more. In some cases, the two sequences that are operatively linked may be separated by at most 1500, 500, 300, 100, 80, 60, 40, 20, 10, 5 bp, or less.

The term “promoter” may be used herein to refer to the regulatory DNA region which controls transcription or expression of a gene and which can be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “2A peptide” may refer to a class of viral oligopeptides (e.g., 18-22 amino-acid (aa)-long viral oligopeptides) that mediate “cleavage” of polypeptides during translation in cells (e.g., eukaryotic cells). The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (Thosea asigna virus 2A) were also identified. The mechanism of 2A-mediated “self-cleavage” is believed to be ribosome skipping the formation of a glycyl-prolyl peptide bond at the C-terminus of the 2A sequence.

Chimeric Polypeptides

In an aspect, the present disclosure provides a method of regulating expression of a target polynucleotide in a cell. The method may comprise (a) expressing a system in the cell, wherein the cell comprises a receptor (e.g., an endogenous receptor) having a ligand binding domain (i.e., a stimulant binding domain) specific for a ligand (e.g., a stimulant); and (b) contacting the cell with the ligand that binds specifically the ligand binding domain. The system expressed in the cell may comprise a first chimeric polypeptide and a second chimeric polypeptide that are activatable upon the contacting step (b). The receptor may be an endogenous receptor or exogenous receptor. The ligand may be a small molecule, polynucleotide, polypeptide, protein, antibody, ligand and/or receptor from another cell, etc.

One of the first and second chimeric polypeptides can comprise a gene modulating polypeptide (GMP) comprising an actuator moiety linked to a cleavage recognition site. The actuator moiety may be capable of regulating the expression of the target polynucleotide in the cell. The other of the first and second chimeric polypeptides may comprise a cleavage moiety capable of cleaving the cleavage recognition site of the GMP.

Upon the contacting of the cell by the ligand that binds specifically the ligand binding domain of the receptor (e.g., endogenous receptor), the first and second chimeric polypeptides may be activated such that the cleavage moiety cleaves the cleavage recognition site and releases the actuator moiety from the GMP, thereby regulating the expression of the target polynucleotide in the cell. In some cases, the cleavage moiety may cleave the cleavage recognition site and release the actuator moiety from the GMP in an amount sufficient to regulate the expression of the target polynucleotide in the cell.

In some cases, the first and second chimeric polypeptides may be activatable upon the contacting step (b) to form a signaling complex of the receptor. In some cases, forming a signaling complex of the receptor may comprise complexing (e.g., direct or indirect complexing) between (1) at least one of the first and second chimeric polypeptides and (2): (i) the receptor, (ii) a co-receptor of the receptor, and/or (iii) a signaling moiety (e.g., a downstream signaling moiety) of the receptor and/or the co-receptor. The co-receptor of the receptor may or may not bind the receptor. The signaling moiety may or may not bind the receptor. The downstream signaling moiety of the receptor and/or its co-receptor may be an adaptor protein of the receptor and/or the its co-receptor, kinase, hydrolase (e.g., lipase, phosphatase, glycosidase, peptidase, nucleosidase, etc.), nucleotide exchange factor, an adaptor protein thereof, a fragment thereof, or a combination thereof. In some cases, complexing can be covalent (e.g., disulfide bond) or non-covalent (e.g., hydrogen bond).

In some cases, when forming the signaling complex of the receptor, an interaction between the first and second chimeric polypeptides may be direct and/or indirect. In a direct interaction between the first and second chimeric polypeptides, at least one of the first and second chimeric polypeptides may be configured to directly bind (e.g., via covalent and/or non-covalent interactions) to the other of the first and second chimeric polypeptides. The direct interaction may be sufficient to induce or promote action of the cleavage moiety to cleave and release (or recognize, cleave, and release) the actuator moiety. In an example, one of the first and second chimeric polypeptide sequences may comprise a binding sequence (e.g., an adaptor polypeptide sequence) configured to bind to at least a portion (e.g., an intracellular portion, a cellular signaling domain, etc.) of the other of the first and second chimeric polypeptides. In an indirect interaction between the first and second chimeric polypeptides, the first and second chimeric polypeptides may be configured to be brought closer to each other (e.g., one is recruited towards the other, the first and second chimeric polypeptides become in proximity to each other, etc.) without any direct binding between each other upon the contacting of the cell with the stimulant, relative to without the contacting of the cell with the stimulant. The indirect interaction may be sufficient to induce or promote action of the cleavage moiety to cleave and release (or recognize, cleave, and release) the actuator moiety. In some examples, the first and second chimeric polypeptides may be configured to bind different portions of (i) the receptor, (ii) the co-receptor of the receptor, and/or (iii) the downstream signaling moiety of the receptor and/or the co-receptor. In an example, the receptor may be a T cell receptor (TCR), and the first and second chimeric polypeptides may bind different portions (e.g., different intracellular portions) of the TCR. In another example, the receptor may be the TCR, and the first and second chimeric polypeptides may bind different portions of Linker for activation of T cells (LAT) that is recruited as part of a signaling cascade of the TCR upon activation of the TCR by a stimulant. In a different example, the receptor may be the TCR, and one of the first and second chimeric polypeptides may bind a portion (e.g., an intracellular portion) of the TCR, while the other of the first and second chimeric polypeptides bind a portion of the LAT.

In some cases, at least one of the first and second chimeric polypeptides may not bind with the ligand. In an example, both of the first and second chimeric polypeptides may not bind with the ligand. Alternatively or in addition to, at least one of the first and second chimeric polypeptides may bind with the ligand. In an example, both of the first and second chimeric polypeptides may bind with the ligand.

The first chimeric polypeptide may be a transmembrane protein or an intracellular protein. The second chimeric polypeptide may be a transmembrane protein or an intracellular protein. In some cases, the receptor (e.g., endogenous receptor) may be a transmembrane receptor or an intracellular receptor.

In some cases, the first chimeric polypeptide may comprise a first adaptor moiety that is activatable to bind (1) a first intracellular domain of the receptor, or (2) a first downstream signaling moiety of the receptor. In some cases, the second chimeric polypeptide may comprise a second adaptor moiety that is activatable to bind (1) a second intracellular domain of the receptor, (2) a second downstream signaling moiety of the receptor, or (3) the first adaptor moiety in an activated state.

In some cases, the first adaptor moiety may be activatable to bind the first intracellular domain of the receptor, wherein the second adaptor moiety may be activatable to bind the second intracellular domain of the receptor, and the first and second intracellular domains of the endogenous receptor may be the same or different. In some cases, the first adaptor moiety may be activatable to bind the first intracellular domain of the receptor, and the second adaptor moiety may be activatable to bind the second downstream signaling moiety of the receptor. In some cases, the first adaptor moiety may be activatable to bind the first intracellular domain of the receptor, and the second adaptor moiety may b e activatable to bind the first adaptor moiety in the activated state. In some cases, the first adaptor moiety may be activatable to bind the first downstream signaling moiety of the receptor, the second adaptor moiety may be activatable to bind the second downstream signaling moiety of the receptor, and the first and second downstream signaling moieties of the receptor may be the same or different. In some cases, the first adaptor moiety may be activatable to bind the first downstream signaling moiety of the receptor, and the second adaptor moiety may be activatable to bind the first adaptor moiety in the activated state.

In some cases, the first adaptor moiety and/or the second adaptor moiety may comprise an adaptor protein of the receptor (e.g., endogenous receptor), kinase, hydrolase, nucleotide exchange factor, an adaptor protein thereof, a fragment thereof, or a combination thereof. The hydrolase may be selected from the group consisting of lipase, phosphatase, glycosidase, peptidase, and nucleosidase.

In some cases, the first chimeric polypeptide may comprise the GMP, and the second chimeric polypeptide may comprise the cleavage moiety. In some cases, the second chimeric polypeptide may comprise the GMP, and the first chimeric polypeptide may comprise the cleavage moiety.

In some cases, the receptor may be a T cell receptor (TCR), comprising TCRA, TCRB, TCRG, and/or TCRD. In an example, the cell may comprise TCRA and TCRB, which may form an alpha beta TCR complex. A cell comprising the alpha beta TCR complex may be referred to as an alpha beta cell (e.g., an alpha beta T cell). In another example, the cell may comprise TCRG and TCRD, which may form a gamma delta TCR complex. A cell comprising the gamma delta TCR complex may be referred to as a gamma delta cell (e.g., a gamma delta T cell). The TCR may comprise a co-receptor of TCR, such as CD3, CD4, and/or CD8. The CD3 may comprise CD3E, CD3D, CD3G, and/or CD3Z. In some cases, an intracellular domain of CD3 may comprise at least one immunoreceptor tyrosine-based activation motif (ITAM). In some cases, the first adaptor moiety and/or the second adaptor moiety may comprise LCK, FYN, ZAP-70, LAT, SLP76, ITK, PLC-γ, VAV1, NCK, GADS, GRB2, PI3K, a fragment thereof, or a combination thereof (see Schwartzberg et al. Nature Reviews Immunology. 2005 May; 5(4):284-95 or Abraham et a. Nature Reviews Immunology. 2004 May; 4(4): 301-8).

An adaptor moiety as disclosed herein can be an adaptor protein of the endogenous receptor, kinase, phosphatase, nucleotide exchange factor, an adaptor protein thereof, a fragment thereof, or a combination thereof.

In some cases, the receptor may comprise at least a portion of a C-type lectin-like receptor, such as, for example, a CD94 family receptor. Examples of the CD95 family receptors can include NKG2A, NKG2B, NKG2C, NKG2D, NKG2E, NKG2F, and NKG2G. In such a case, the first adaptor moiety and/or the second adaptor moiety may comprise at least a portion of a signaling adaptor (e.g., a transmembrane signaling adaptor) of the C-type lectin-like receptor. In some examples, the receptor may be NKG2D comprising NKG2D-L and NKG2D-S, and a respective ligand (i.e., NKG2DL) that binds a NKG2DL binding domain and activates NKG2D signaling may include, but are not limited to, MICA, MICB, and the RAET1/ULBP family (e.g., RAET1 E/ULBP4, RAET1G/ULBP5, RAET1 H/ULBP2, RAET1/ULBP1, RAET1 L/ULBP6, and RAET1 N/ULBP3). In some cases, the receptor may be NKG2D, and the first adaptor moiety and/or the second adaptor moiety may comprise DAP10, DAP12, PI3K, GRB2, VAV1, SYK, ZAP-70, a fragment thereof, or a combination thereof (see Zafirova et al. Cellular and Molecular Life Sciences. 2011 August; 68(21):3519-29 or Sheppard et al. Frontiers in Immunology. 2018 August; 9(1808):1-19).

In some cases, the receptor may be a Toll-like receptor (TLR) selected from the group consisting of: TLR1, TLR2, TLR3, TLR4, TLR5, TLR6, TLR7, TLR8, TLR9, TLR10, TLR11, TLR12, and TLR13. Examples of ligands for the TLR may include, but are not limited to, a lipopolysaccharide, lipoprotein, triacylated lipopeptides, peptidoglycan, flagella, single-stranded RNA, double-stranded RNA, CpG DNA, profilin, and ribosomal RNA. At least one of the ligands for the TLR may be originated from a bacteria or virus. In some cases, the first adaptor moiety and/or the second adaptor moiety may comprise MyD88, Tube, Pelle, TIRAP, TRIF, TRAM, IRAK1, TRAK4, TRAF6, TAK1, TBK1, RIPK1, PI3K, IKK, a fragment thereof, or a combination thereof (see O'Neill et al. Nature Reviews Immunology. 2013 June; 13(6):453-60 or Wang et al. Frontiers in Immunology. 2014 July; 5(367):1-11).

In another aspect, the present disclosure provides a system for regulating expression of a target polynucleotide in a cell. The system may comprise a first chimeric polypeptide and a second chimeric polypeptide. One of the first and second chimeric polypeptides may comprise a gene modulating polypeptide (GMP) comprising an actuator moiety linked to a cleavage recognition site. The actuator moiety may be capable of regulating the expression of the target polynucleotide in the cell. The other of the first and second chimeric polypeptides may comprise a cleavage moiety capable of cleaving the cleavage recognition site of the GMP. The cell may comprise a receptor having a ligand binding domain specific for a ligand. The first and second chimeric polypeptides may be activatable upon contacting of the cell by the ligand that binds specifically the ligand binding domain of the endogenous receptor. Upon the contacting of the cell by the ligand, the first and second chimeric polypeptides may be activated such that the cleavage moiety cleaves the cleavage recognition site and releases the actuator moiety from the GMP, thereby regulating the expression of the target polynucleotide in the cell. In some cases, the cleavage moiety may cleave the cleavage recognition site and release the actuator moiety from the GMP in an amount sufficient to regulate the expression of the target polynucleotide in the cell. The receptor may be an endogenous receptor or exogenous receptor.

In some cases, the first and second chimeric polypeptides may be activatable upon the contacting to form a signaling complex of the receptor. In some cases, forming a signaling complex of the receptor may comprise complexing (e.g., direct or indirect complexing) between (1) at least one of the first and second chimeric polypeptides and (2): (i) the receptor, (ii) a co-receptor of the receptor, and/or (iii) a signaling moiety (e.g., a downstream signaling moiety) of the receptor and/or the co-receptor. In some cases, complexing can be covalent (e.g., disulfide bond) or non-covalent (e.g., hydrogen bond).

In some cases, the first and second chimeric polypeptides may bind the ligand. In some cases, the first and second chimeric polypeptides may not bind the ligand.

In some cases, the first chimeric polypeptide may be a transmembrane protein or an intracellular protein. In some cases, the second chimeric polypeptide may be a transmembrane protein or an intracellular protein. The receptor may be a transmembrane receptor or an intracellular receptor.

In some cases, the first chimeric polypeptide may comprise a first adaptor moiety that is activatable to bind (1) a first intracellular domain of the endogenous receptor, or (2) a first downstream signaling moiety of the receptor. In some cases, the second chimeric polypeptide may comprise a second adaptor moiety that is activatable to bind (1) a second intracellular domain of the receptor, (2) a second downstream signaling moiety of the receptor, or (3) the first adaptor moiety in an activated state. In some cases, the first adaptor moiety may be activatable to bind the first intracellular domain of the receptor, the second adaptor moiety may be activatable to bind the second intracellular domain of the receptor, and the first and second intracellular domains of the receptor may be the same or different. In some cases, the first adaptor moiety may be activatable to bind the first intracellular domain of the receptor, and the second adaptor moiety may be activatable to bind the second downstream signaling moiety of the receptor. In some cases, the first adaptor moiety may be activatable to bind the first intracellular domain of the receptor, and the second adaptor moiety may be activatable to bind the first adaptor moiety in the activated state. In some cases, the first adaptor moiety may be activatable to bind the first downstream signaling moiety of the receptor, the second adaptor moiety may be activatable to bind the second downstream signaling moiety of the receptor, and the first and second downstream signaling moieties of the receptor may be the same or different. In some cases, the first adaptor moiety may be activatable to bind the first downstream signaling moiety of the receptor, and wherein the second adaptor moiety may be activatable to bind the first adaptor moiety in the activated state.

In some cases, the first adaptor moiety and/or the second adaptor moiety may comprise an adaptor protein of the receptor (e.g., an endogenous receptor), kinase, hydrolase, phosphatase, nucleotide exchange factor, an adaptor protein thereof, a fragment thereof, or a combination thereof. The hydrolase may be selected from the group consisting of lipase, phosphatase, glycosidase, peptidase, and nucleosidase.

In some cases, the first chimeric polypeptide may comprise the GMP, and wherein the second chimeric polypeptide may comprise the cleavage moiety. In some cases, the second chimeric polypeptide may comprise the GMP, and the first chimeric polypeptide comprises the cleavage moiety.

In some cases, the endogenous receptor may be a T cell receptor (TCR), comprising TCRA, TCRB, TCRG, and/or TCRD. The TCR may comprise a co-receptor of TCR, comprising CD3, CD4, and/or CD8. The CD3 may comprise CD3E, CD3D, CD3G, and/or CD3Z. The intracellular domain of CD3 may comprise at least one immunoreceptor tyrosine-based activation motif (ITAM). The first adaptor moiety and/or the second adaptor moiety may comprise LCK, FYN, ZAP-70, LAT, SLP76, ITK, PLC-γ, VAV1, NCK, GADS, GRB2, PI3K, a fragment thereof, or a combination thereof.

In some cases, the endogenous receptor may be NKG2D, comprising NKG2D-L and NKG2D-S. The first adaptor moiety and/or the second adaptor moiety may comprise DAP10, DAP12, PI3K, GRB2, VAV1, SYK, ZAP-70, a fragment thereof, or a combination thereof.

In some cases, the endogenous receptor may be a Toll-like receptor (TLR) selected from the group consisting of: TLR1, TLR2, TLR3, TLR4, TLR5, TLR6, TLR7, TLR8, TLR9, TLR10, TLR11, TLR12, and TLR13. The first adaptor moiety and/or the second adaptor moiety may comprise MyD88, Tube, Pelle, TIRAP, TRIF, TRAM, IRAK1, TRAK4, TRAF6, TAK1, TBK1, RIPK1, PI3K, IKK, a fragment thereof, or a combination thereof.

In another aspect, the present disclosure provides a method of regulating expression of a target polynucleotide in a cell. The method may comprise (a) expressing a system in the cell, wherein the cell comprises a receptor having a ligand binding domain specific for a ligand; and (b) contacting the cell with the ligand that binds specifically the ligand binding domain. The system expressed in the cell may comprise a first chimeric polypeptide and a second chimeric polypeptide that are activatable upon the contacting step (b). One of the first and second chimeric polypeptides may comprise a gene modulating polypeptide (GMP) comprising an actuator moiety linked to a cleavage recognition site, which actuator moiety is capable of regulating the expression of the target polynucleotide in the cell. The other of the first and second chimeric polypeptides may comprise a cleavage moiety capable of cleaving the cleavage recognition site of the GMP. The at least one of the first and second chimeric polypeptides may not be in direct contact with the receptor. Upon the contacting of the cell by the ligand that binds specifically the ligand binding domain of the receptor, the first and second chimeric polypeptides may be activated such that the cleavage moiety cleaves the cleavage recognition site and releases the actuator moiety from the GMP, thereby regulating the expression of the target polynucleotide in the cell. In some cases, the cleavage moiety may cleave the cleavage recognition site and release the actuator moiety from the GMP in an amount sufficient to regulate the expression of the target polynucleotide in the cell.

The receptor may be an endogenous receptor or exogenous receptor. The exogenous receptor may comprise a chimeric polypeptide. The chimeric polypeptide may comprise a chimeric antigen receptor. The receptor may be a heterologous receptor.

The at least one of the first and second chimeric polypeptides may lack an ability to be in direct contact (e.g., bind) with the receptor. The at least one of the first and second chimeric polypeptides may not be in direct contact with the receptor prior to, during, and/or subsequent to the contacting of the cell by the ligand. The at least one of the first and second chimeric polypeptides may not be in direct contact with the receptor prior to, during, and subsequent to the contacting of the cell by the ligand. In some cases, only one of the first and second chimeric polypeptides is not in direct contact with the receptor.

In some cases, the first and second chimeric polypeptides are activatable upon the contacting step (b) to form a signaling complex of the receptor. Forming a signaling complex of the receptor may comprise complexing (e.g., direct or indirect complexing) between (1) at least one of the first and second chimeric polypeptides and (2): (i) the receptor, (ii) a co-receptor of the receptor, and/or (iii) a signaling moiety (e.g., a downstream signaling moiety) of the receptor and/or the co-receptor. In some cases, complexing can be covalent (e.g., disulfide bond) or non-covalent (e.g., hydrogen bond).

In some cases, the first and second chimeric polypeptides bind the ligand. In some cases, the first and second chimeric polypeptides do not bind the ligand.

The first chimeric polypeptide may be a transmembrane protein or an intracellular protein. The second chimeric polypeptide may be a transmembrane protein or an intracellular protein. The receptor may be a transmembrane receptor or an intracellular receptor.

In some cases, the first chimeric polypeptide comprises a first adaptor moiety that is activatable to bind (1) an intracellular domain of the receptor, or (2) a first downstream signaling moiety of the receptor. In some cases, the second chimeric polypeptide comprises a second adaptor moiety that is activatable to bind (1) a second downstream signaling moiety of the receptor, or (2) the first adaptor moiety in an activated state.

In some cases, the first adaptor moiety is activatable to bind the intracellular domain of the receptor, and the second adaptor moiety is activatable to bind the second downstream signaling moiety of the receptor. In some cases, the first adaptor moiety is activatable to bind the first intracellular domain of the receptor, and the second adaptor moiety is activatable to bind the first adaptor moiety in the activated state. In some cases, the first adaptor moiety is activatable to bind the first downstream signaling moiety of the receptor, the second adaptor moiety is activatable to bind the second downstream signaling moiety of the receptor, and the first and second downstream signaling moieties of the receptor are the same or different. In some cases, the first adaptor moiety is activatable to bind the first downstream signaling moiety of the receptor, and the second adaptor moiety is activatable to bind the first adaptor moiety in the activated state.

In some cases, the receptor is a T cell receptor (TCR), comprising TCRA, TCRB, TCRG, and/or TCRD. The TCR may comprise a co-receptor of TCR, comprising CD3, CD4, and/or CD8. The CD3 may comprise CD3E, CD3D, CD3G, and/or CD3Z. An intracellular domain of CD3 may comprise at least one immunoreceptor tyrosine-based activation motif (ITAM). The first adaptor moiety and/or the second adaptor moiety may comprise LCK, FYN, ZAP-70, LAT, SLP76, ITK, PLC-γ, VAV1, NCK, GADS, GRB2, PI3K, a fragment thereof, or a combination thereof.

In some cases, the receptor is NKG2D, comprising NKG2D-L and NKG2D-S. The receptor may be NKG2D, and the first adaptor moiety and/or the second adaptor moiety may comprise DAP10, DAP12, PI3K, GRB2, VAV1, SYK, ZAP-70, a fragment thereof, or a combination thereof.

In some cases, the receptor is a Toll-like receptor (TLR) selected from the group consisting of: TLR1, TLR2, TLR3, TLR4, TLR5, TLR6, TLR7, TLR8, TLR9, TLR10, TLR11, TLR12, and TLR13. The first adaptor moiety and/or the second adaptor moiety may comprise MyD88, Tube, Pelle, TIRAP, TRIF, TRAM, IRAK1, TRAK4, TRAF6, TAK1, TBK1, RIPK1, PI3K, IKK, a fragment thereof, or a combination thereof.

In another aspect, the present disclosure provides a system for regulating expression of a target polynucleotide in a cell. The system may comprise a first chimeric polypeptide and a second chimeric polypeptide. One of the first and second chimeric polypeptides may comprise a gene modulating polypeptide (GMP) comprising an actuator moiety linked to a cleavage recognition site. The actuator moiety may be capable of regulating the expression of the target polynucleotide in the cell. The other of the first and second chimeric polypeptides may comprise a cleavage moiety capable of cleaving the cleavage recognition site of the GMP. The cell may comprise a receptor having a ligand binding domain specific for a ligand. The first and second chimeric polypeptides may be activatable upon contacting of the cell by the ligand that binds specifically the ligand binding domain of the receptor. The at least one of the first and second chimeric polypeptides may not be in direct contact with the receptor. Upon the contacting of the cell by the ligand, the first and second chimeric polypeptides may be activated such that the cleavage moiety cleaves the cleavage recognition site and releases the actuator moiety from the GMP, thereby regulating the expression of the target polynucleotide in the cell. In some cases, the cleavage moiety may cleave the cleavage recognition site and release the actuator moiety from the GMP in an amount sufficient to regulate the expression of the target polynucleotide in the cell.

The receptor may comprise an endogenous receptor or an exogenous receptor. The exogenous receptor may comprise a chimeric polypeptide. The chimeric polypeptide may comprise a chimeric antigen receptor. The receptor may be a heterologous receptor.

The at least one of the first and second chimeric polypeptides may lack an ability to be in direct contact (e.g., bind) with the receptor. The at least one of the first and second chimeric polypeptides may not be in direct contact with the receptor prior to, during, and/or subsequent to the contacting of the cell by the ligand. The at least one of the first and second chimeric polypeptides may not be in direct contact with the receptor prior to, during, and subsequent to the contacting of the cell by the ligand. In some cases, only one of the first and second chimeric polypeptides is not in direct contact with the receptor.

In some cases, the first and second chimeric polypeptides are activatable upon the contacting to form a signaling complex of the receptor. Forming a signaling complex of the receptor may comprise complexing (e.g., direct or indirect complexing) between (1) at least one of the first and second chimeric polypeptides and (2): (i) the receptor, (ii) a co-receptor of the receptor, and/or (iii) a signaling moiety (e.g., a downstream signaling moiety) of the receptor and/or the co-receptor. In some cases, complexing can be covalent (e.g., disulfide bond) or non-covalent (e.g., hydrogen bond).

In some cases, the first and second chimeric polypeptides may bind the ligand. In some cases, the first and second chimeric polypeptides do not bind the ligand.

In some cases, the first chimeric polypeptide is a transmembrane protein or an intracellular protein. In some cases, the second chimeric polypeptide is a transmembrane protein or an intracellular protein. The receptor may be a transmembrane receptor or an intracellular receptor.

In some cases, the first chimeric polypeptide comprises a first adaptor moiety that is activatable to bind (1) an intracellular domain of the receptor, or (2) a first downstream signaling moiety of the receptor. In some cases, the second chimeric polypeptide comprises a second adaptor moiety that is activatable to bind (1) a second downstream signaling moiety of the receptor, or (2) the first adaptor moiety in an activated state.

In some cases, the first adaptor moiety is activatable to bind the intracellular domain of the receptor, and the second adaptor moiety is activatable to bind the second downstream signaling moiety of the receptor. In some cases, the first adaptor moiety is activatable to bind the first intracellular domain of the receptor, and the second adaptor moiety is activatable to bind the first adaptor moiety in the activated state. In some cases, the first adaptor moiety is activatable to bind the first downstream signaling moiety of the receptor, the second adaptor moiety is activatable to bind the second downstream signaling moiety of the receptor, and the first and second downstream signaling moieties of the receptor are the same or different. In some cases, the first adaptor moiety is activatable to bind the first downstream signaling moiety of the receptor, and the second adaptor moiety is activatable to bind the first adaptor moiety in the activated state.

In some cases, the receptor is a T cell receptor (TCR), comprising TCRA, TCRB, TCRG, and/or TCRD. The TCR may comprise a co-receptor of TCR, comprising CD3, CD4, and/or CD8. The CD3 may comprise CD3E, CD3D, CD3G, and/or CD3Z. An intracellular domain of CD3 may comprise at least one immunoreceptor tyrosine-based activation motif (ITAM). The first adaptor moiety and/or the second adaptor moiety may comprise LCK, FYN, ZAP-70, LAT, SLP76, ITK, PLC-γ, VAV1, NCK, GADS, GRB2, PI3K, a fragment thereof, or a combination thereof.

In some cases, the receptor is NKG2D, comprising NKG2D-L and NKG2D-S. The receptor may be NKG2D, and the first adaptor moiety and/or the second adaptor moiety comprises DAP10, DAP12, PI3K, GRB2, VAV1, SYK, ZAP-70, a fragment thereof, or a combination thereof.

In some cases, the receptor is a Toll-like receptor (TLR) selected from the group consisting of: TLR1, TLR2, TLR3, TLR4, TLR5, TLR6, TLR7, TLR8, TLR9, TLR10, TLR11, TLR12, and TLR13. The first adaptor moiety and/or the second adaptor moiety may comprise MyD88, Tube, Pelle, TIRAP, TRIF, TRAM, IRAK1, TRAK4, TRAF6, TAK1, TBK1, RIPK1, PI3K, IKK, a fragment thereof, or a combination thereof.

In another aspect, the present disclosure provides a method of regulating expression of a target polynucleotide in a cell. The method may comprise (a) expressing a system in the cell, wherein the cell comprises a receptor having a ligand binding domain specific for a ligand; and (b) contacting the cell with the ligand that binds specifically the ligand binding domain. The system expressed in the cell may comprise a first chimeric polypeptide and a second chimeric polypeptide that are activatable upon the contacting step (b). One of the first and second chimeric polypeptides may comprise a gene modulating polypeptide (GMP) comprising an actuator moiety linked to a cleavage recognition site. The actuator moiety may be capable of regulating the expression of the target polynucleotide in the cell. The other of the first and second chimeric polypeptides may comprise a cleavage moiety capable of cleaving the cleavage recognition site of the GMP. The first and second chimeric polypeptides may not be in direct contact with the receptor but may be in association with signaling of the receptor. Upon the contacting of the cell by the ligand that binds specifically the ligand binding domain of the receptor, the first and second chimeric polypeptides may be activated such that the cleavage moiety cleaves the cleavage recognition site and releases the actuator moiety from the GMP, thereby regulating the expression of the target polynucleotide in the cell. In some cases, the cleavage moiety may cleave the cleavage recognition site and release the actuator moiety from the GMP in an amount sufficient to regulate the expression of the target polynucleotide in the cell.

In some cases, the receptor comprises an endogenous receptor or an exogenous receptor. The exogenous receptor may comprise a chimeric polypeptide. The chimeric polypeptide may comprise a chimeric antigen receptor. The receptor may be a heterologous receptor.

Both of the first and second chimeric polypeptides may lack an ability to be in direct contact (e.g., bind) with the receptor. Both of the first and second chimeric polypeptides may not be in direct contact with the receptor prior to, during, and/or subsequent to the contacting of the cell by the ligand. Both of the first and second chimeric polypeptides may not be in direct contact with the receptor prior to, during, and subsequent to the contacting of the cell by the ligand. In some cases, both of the first and second chimeric polypeptides is not in direct contact with the receptor.

The first and second chimeric polypeptides may be activatable upon the contacting step (b) to form a signaling complex of the receptor. Forming a signaling complex of the receptor may comprise indirect complexing between (1) at least one of the first and second chimeric polypeptides and (2) the receptor. Alternatively or in addition to, forming the signaling complex of the receptor may comprise complexing (e.g., direct or indirect complex) between (1) at least one of the first and second chimeric polypeptides and (2): (ii) a co-receptor of the receptor, and/or (iii) a signaling moiety (e.g., a downstream signaling moiety) of the receptor and/or the co-receptor. In some cases, complexing can be covalent (e.g., disulfide bond) or non-covalent (e.g., hydrogen bond).

In some cases, the first and second chimeric polypeptides may bind the ligand. The first and second chimeric polypeptides may not bind the ligand.

In some cases, the first chimeric polypeptide is a transmembrane protein or an intracellular protein. In some cases, the second chimeric polypeptide is a transmembrane protein or an intracellular protein. The receptor may be a transmembrane receptor or an intracellular receptor.

In some cases, the first chimeric polypeptide comprises a first adaptor moiety that is activatable to bind a first downstream signaling moiety of the receptor. In some cases, the second chimeric polypeptide comprises a second adaptor moiety that is activatable to bind (A) a second downstream signaling moiety of the receptor, or (B) the first adaptor moiety in an activated state.

In some cases, the first adaptor moiety is activatable to bind the first downstream moiety of the receptor, the second adaptor moiety is activatable to bind the second downstream moiety of the receptor, and the first and second downstream moieties of the receptor are the same or different. In some cases, the first adaptor moiety is activatable to bind the first downstream signaling moiety of the receptor, and the second adaptor moiety is activatable to bind the first adaptor moiety in the activated state.

In some cases, the receptor is a T cell receptor (TCR), comprising TCRA, TCRB, TCRG, and/or TCRD. The TCR may comprise a co-receptor of TCR, comprising CD3, CD4, and/or CD8. The CD3 may comprise CD3E, CD3D, CD3G, and/or CD3Z. An intracellular domain of CD3 may comprise at least one immunoreceptor tyrosine-based activation motif (ITAM). The first adaptor moiety and/or the second adaptor moiety may comprise LAT, SLP76, ITK, PLC-γ, VAV1, NCK, GADS, GRB2, PI3K, a fragment thereof, or a combination thereof.

In some cases, the receptor is NKG2D, comprising NKG2D-L and NKG2D-S. The receptor may be NKG2D, and the first adaptor moiety and/or the second adaptor moiety may comprise PI3K, GRB2, VAV1, SYK, ZAP-70, a fragment thereof, or a combination thereof.

In some cases, the receptor is a Toll-like receptor (TLR) selected from the group consisting of: TLR1, TLR2, TLR3, TLR4, TLR5, TLR6, TLR7, TLR8, TLR9, TLR10, TLR11, TLR12, and TLR13. The first adaptor moiety and/or the second adaptor moiety may comprise Tube, Pelle, IRAK1, TRAK4, TRAF6, TAK1, TBK1, RIPK1, PI3K, IKK, a fragment thereof, or a combination thereof.

In another aspect, the present disclosure provides a system for regulating expression of a target polynucleotide in a cell. The system may comprise a first chimeric polypeptide and a second chimeric polypeptide. One of the first and second chimeric polypeptides may comprise a gene modulating polypeptide (GMP) comprising an actuator moiety linked to a cleavage recognition site. The actuator moiety may be capable of regulating the expression of the target polynucleotide in the cell. The other of the first and second chimeric polypeptides may comprise a cleavage moiety capable of cleaving the cleavage recognition site of the GMP. The cell may comprise a receptor having a ligand binding domain specific for a ligand. The first and second chimeric polypeptides may be activatable upon contacting of the cell by the ligand that binds specifically the ligand binding domain of the receptor. The first and second chimeric polypeptides may not be in direct contact with the receptor but may be in association with signaling of the receptor. Upon the contacting of the cell by the ligand, the first and second chimeric polypeptides may be activated such that the cleavage moiety cleaves the cleavage recognition site and releases the actuator moiety from the GMP, thereby regulating the expression of the target polynucleotide in the cell. In some cases, the cleavage moiety may cleave the cleavage recognition site and release the actuator moiety from the GMP in an amount sufficient to regulate the expression of the target polynucleotide in the cell.

In some cases, the receptor comprises an endogenous receptor or an exogenous receptor. The exogenous receptor may comprise a chimeric polypeptide. The chimeric polypeptide may comprise a chimeric antigen receptor. The receptor may be a heterologous receptor.

Both of the first and second chimeric polypeptides may lack an ability to be in direct contact (e.g., bind) with the receptor. Both of the first and second chimeric polypeptides may not be in direct contact with the receptor prior to, during, and/or subsequent to the contacting of the cell by the ligand. Both of the first and second chimeric polypeptides may not be in direct contact with the receptor prior to, during, and subsequent to the contacting of the cell by the ligand. In some cases, both of the first and second chimeric polypeptides is not in direct contact with the receptor.

The first and second chimeric polypeptides may be activatable upon the contacting to form a signaling complex of the receptor. Forming a signaling complex of the receptor may comprise indirect complexing between (1) at least one of the first and second chimeric polypeptides and (2) the receptor. Alternatively or in addition to, forming the signaling complex of the receptor may comprise complexing (e.g., direct or indirect complex) between (1) at least one of the first and second chimeric polypeptides and (2): (ii) a co-receptor of the receptor, and/or (iii) a signaling moiety (e.g., a downstream signaling moiety) of the receptor and/or the co-receptor. In some cases, complexing can be covalent (e.g., disulfide bond) or non-covalent (e.g., hydrogen bond).

In some cases, the first and second chimeric polypeptides may bind the ligand. In some cases, the first and second chimeric polypeptides do not bind the ligand.

In some cases, the first chimeric polypeptide is a transmembrane protein or an intracellular protein. In some cases, the second chimeric polypeptide is a transmembrane protein or an intracellular protein. The receptor may be a transmembrane receptor or an intracellular receptor. The first chimeric polypeptide may comprise a first adaptor moiety that is activatable to bind a first downstream signaling moiety of the receptor. The second chimeric polypeptide may comprise a second adaptor moiety that is activatable to bind (A) a second downstream signaling moiety of the receptor, or (B) the first adaptor moiety in an activated state.

In some cases, the first adaptor moiety is activatable to bind the first downstream moiety of the receptor, the second adaptor moiety is activatable to bind the second downstream moiety of the receptor, and the first and second downstream moieties of the receptor are the same or different. In some cases, the first adaptor moiety is activatable to bind the first downstream signaling moiety of the receptor, and the second adaptor moiety is activatable to bind the first adaptor moiety in the activated state.

In some cases, the receptor is a T cell receptor (TCR), comprising TCRA, TCRB, TCRG, and/or TCRD. The TCR may comprise a co-receptor of TCR, comprising CD3, CD4, and/or CD8. The CD3 may comprise CD3E, CD3D, CD3G, and/or CD3Z. An intracellular domain of CD3 may comprise at least one immunoreceptor tyrosine-based activation motif (ITAM). The first adaptor moiety and/or the second adaptor moiety may comprise LAT, SLP76, ITK, PLC-γ, VAV1, NCK, GADS, GRB2, PI3K, a fragment thereof, or a combination thereof.

In some cases, the receptor is NKG2D, comprising NKG2D-L and NKG2D-S. The receptor may be NKG2D, and the first adaptor moiety and/or the second adaptor moiety may comprise PI3K, GRB2, VAV1, SYK, ZAP-70, a fragment thereof, or a combination thereof.

In some cases, the receptor is a Toll-like receptor (TLR) selected from the group consisting of: TLR1, TLR2, TLR3, TLR4, TLR5, TLR6, TLR7, TLR8, TLR9, TLR10, TLR11, TLR12, and TLR13. The first adaptor moiety and/or the second adaptor moiety may comprise Tube, Pelle, IRAK1, TRAK4, TRAF6, TAK1, TBK1, RIPK1, PI3K, IKK, a fragment thereof, or a combination thereof.

Binding Affinity

In some cases, one or more characteristics of a binding affinity (e.g., equilibrium dissociation constant (K_{D}), equilibrium association constant (K_{A}), etc.) between two molecules of interest (e.g., between one of the first and second chimeric polypeptides and a portion of the receptor, between the first and second chimeric polypeptides, etc.) provided herein in the present disclosure may be assessed by techniques such as, for example, enzyme-linked immunosorbent assay (ELISA), surface plasmon resonance (SPR), isothermal titration calorimetry (ITC), fluorescence depolarization, one or more computer simulations, etc.

In some cases, at least one of the first and second chimeric polypeptides may directly complex with at least one of (i) the receptor, (ii) a co-receptor of the receptor, and/or (iii) a signaling moiety (e.g., a downstream signaling moiety) of the receptor and/or the co-receptor, with a K_{D} (wherein K_{D}=K_{off} (i.e., “k_{d}”)/Kₒₙ (i.e., “kₐ”)) of about 10⁻¹⁵ molar (M) to about 10⁻⁵M. In some cases, the at least one of the first and second chimeric polypeptides may directly complex with the at least one of (i) the receptor, (ii) a co-receptor of the receptor, and/or (iii) a signaling moiety of the receptor and/or the co-receptor, with a K_{D} of at least about 10⁻¹⁵M, 10⁻¹⁴M, 10⁻¹³M, 10⁻¹²M, 10⁻¹¹M, 10⁻¹⁰M, 10⁻⁹M, 10⁻⁸M, 10⁻⁷M, 10⁻⁶M, 10⁻⁵M, or more. In some cases, the at least one of the first and second chimeric polypeptides may directly complex with the at least one of (i) the receptor, (ii) a co-receptor of the receptor, and/or (iii) a signaling moiety of the receptor and/or the co-receptor, with a K_{D} of at most about 10⁻⁵M, 10⁻⁶M, 10⁻⁷M, 10⁻⁸M, 10⁻⁹M, 10⁻¹⁰M, 10⁻¹¹M, 10⁻¹²M, 10⁻¹³M, 10⁻¹⁴M, 10⁻¹⁵M, or less.

In some cases, the first and second chimeric polypeptides may directly complex with each other with a K_{D} of about 10⁻¹⁵ molar (M) to about 10⁻⁵M. In some cases, the first and second chimeric polypeptides may directly complex with each other with a K_{D} of at least about 10⁻¹⁵M, 10⁻¹⁴M, 10⁻¹³M, 10⁻¹²M, 10⁻¹¹M, 10⁻¹⁰M, 10⁻⁹M, 10⁻⁸M, 10⁻⁷M, 10⁻⁶M, 10⁻⁵M, or more. In some cases, the first and second chimeric polypeptides may directly complex with each other with a K_{D} of at most about 10⁻⁵M, 10⁻⁶M, 10⁻⁷M, 10⁻⁸M, 10⁻⁹M, 10⁻¹⁰M, 10⁻¹¹M, 10⁻¹²M, 10⁻¹³M, 10⁻¹⁴M, 10⁻¹⁵M, or less.

GMP and Actuator Moiety

The GMP may comprise an actuator moiety that regulates expression of a target polynucleotide in the cell. The target polynucleotide in the cell may encode a target polypeptide. In some cases, the target polypeptide may induce or inhibit proliferation, differentiation, and/or survival of the cell. The actuator moiety can bind to a target polynucleotide to regulate expression and/or activity of a target gene encoded by the target polynucleotide. In some embodiments, the target polynucleotide comprises genomic DNA. In some embodiments, the target polynucleotide comprises a region of a plasmid, for example a plasmid carrying an exogenous gene. In some embodiments, the target polynucleotide comprises RNA, for example mRNA. In some embodiments, the target polynucleotide comprises an endogenous gene or gene product. The actuator moiety can comprise a nuclease (e.g., DNA nuclease and/or RNA nuclease), modified nuclease (e.g., DNA nuclease and/or RNA nuclease) that is nuclease-deficient or has reduced nuclease activity compared to a wild-type nuclease or a variant thereof. The actuator moiety can regulate expression or activity of a gene and/or edit the sequence of a nucleic acid (e.g., a gene and/or gene product). In some embodiments, the actuator moiety comprises a DNA nuclease such as an engineered (e.g., programmable or targetable) DNA nuclease to induce genome editing of a target DNA sequence. In some embodiments, the actuator moiety comprises a RNA nuclease such as an engineered (e.g., programmable or targetable) RNA nuclease to induce editing of a target RNA sequence. In some embodiments, the actuator moiety has reduced or minimal nuclease activity (e.g., dCas). An actuator moiety having reduced or minimal nuclease activity can regulate expression and/or activity of a gene by physical obstruction of a target polynucleotide or recruitment of additional factors effective to suppress or enhance expression of the target polynucleotide. The actuator moiety can physically obstruct the target polynucleotide or recruit additional factors effective to suppress or enhance expression of the target polynucleotide. In some cases, the actuator moiety comprises an activator effective to increase expression of the target polynucleotide. In some embodiments, the actuator moiety comprises a transcriptional activator effective to increase expression of the target polynucleotide. In other cases, the actuator moiety comprises a repressor effective to decrease expression of the target polynucleotide. Non-limiting examples of transcription activators include GAL4, VP16, VP64, p65 subdomain (NFkappaB), and VP64-p65-Rta (VPR). In some embodiments, the actuator moiety comprises a transcriptional repressor effective to decrease expression of the target polynucleotide. Non-limiting examples of transcription repressors include Kruippel associated box (KRAB or SKD), the Mad mSIN3 interaction domain (SID), and the ERF repressor domain (ERD). In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. In some embodiments, the actuator moiety is a nucleic acid-guided actuator moiety. In some embodiments, the actuator moiety is a DNA-guided actuator moiety. In some embodiments, the actuator moiety is an RNA-guided actuator moiety or a variant thereof, which RNA-guided actuator moiety forms a complex with the target polynucleotide. An actuator moiety can regulate expression or activity of a gene and/or edit a nucleic acid sequence, whether exogenous or endogenous.

Any suitable nuclease can be used. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); and any variant thereof. In some cases, the actuator moiety is a CRISPR-associated (Cas) protein or a fragment thereof that substantially lacks DNA cleavage activity (dCas). In some cases, the actuator moiety can be Cas9 and/or Cpf1.

Any target gene can be regulated by the comprising the actuator moiety. It is contemplated that genetic homologues of a gene described herein are covered. For example, a gene can exhibit a certain identity and/or homology to genes disclosed herein. Therefore, it is contemplated that the expression of a gene that exhibits or exhibits at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level) can be regulated. It is also contemplated that the expression of a gene that exhibits or exhibits at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be regulated.

Administration of GMP

In some cases, the administration of the GMP to the cell can comprise treating the cell with a delivery vehicle, which delivery vehicle comprises at least a portion of the GMP and/or a polynucleotide that encodes at least a portion of the GMP. The delivery vehicle may be viral or non-viral. The at least the portion of the GMP and/or the polynucleotide that encodes the at least the portion of the GMP may be attached covalently and/or non-covalently (e.g., ionically, via hydrogen bonds, etc.) to the delivery vehicle. Alternatively or in addition to, the at least the portion of the GMP and/or the polynucleotide that encodes the at least the portion of the GMP may be encapsulated by the delivery vehicle without any physical attachment to the delivery vehicle.

In some cases, the delivery vehicle may comprise a targeting moiety with an affinity to one or more ligands (e.g., a portion of a cell surface receptor, a polysaccharide chain, one or more extracellular proteins) present on or adjacent to the surface of the cell. The targeting moiety may enhance targeting and binding of the delivery vehicle to the cell. The targeting moiety may enhance intracellular entrance, uptake, and/or penetration of the delivery vehicle into the cell. The targeting moiety may be linked (e.g., via covalent and/or a non-covalent bond) to an external surface of the delivery vehicle. The targeting moiety may be a non-natural molecule, at least a portion of a natural molecule, a functional derivative thereof, or a combination thereof. The targeting moiety may be a small molecule, a polynucleotide (e.g., an aptamer), a polypeptide (e.g., an oligopeptide or a protein), an antibody or a functional fragment thereof, a functional derivative thereof, or a combination thereof.

In some cases, the delivery vehicle may not comprise such targeting moiety against the cell.

Examples of the viral delivery vehicle may comprise an adenovirus, a retrovirus, a lentivirus (e.g., a human immunodeficiency virus (HIV)), an adeno-associated virus (AAV), and/or a Herpes simplex virus (HSV). In an example, the viral delivery vehicle may be a retrovirus. The retrovirus may be a gamma-retrovirus selected from the group consisting of: Feline Leukemia Virus (FLV), Feline Sarcoma Virus (Strain Hardy-Zuckerman 4), Finkel-Biskis-Jinkins Murine Sarcoma Virus (FBJMSV), Murine leukemia virus (MLV) (e.g. Friend Murine Leukemia Virus (FMLV), Moloney Murine Leukemia Virus (MMLV), Murine Type C Retrovirus (MTCR)), Gibbon Ape Leukemia Virus (GALV), Koala Retrovirus (KR), Moloney Murine Sarcoma Virus (MMSV), Porcine Endogenous Retrovirus E (PERE), Reticuloendotheliosis Virus (RV), Woolly Monkey Sarcoma Virus (WMSV), Baboon Endogenous Virus Strain M7 (BEVSM7), Murine Osteosarcoma Virus (MOV), Mus Musculus Mobilized Endogenous Polytropic Provirus (MMMEPP), PreXMRV-1, RD114 Retrovirus, Spleen Focus-Forming Virus (SFFV), Abelson murine leukemia virus (AMLV), Murine Stem Cell Virus (MSCV), and variants thereof.

The delivery vehicle may comprise of a nucleotide (e.g., a polynucleotide), an amino acid (e.g., a peptide or polypeptide), a polymer, a metal, a ceramic, a derivative thereof, or a combination thereof. In an example, the delivery vehicle may comprise of a diamond nanoparticle (“nanodiamonds”), a gold nanoparticle, a silver nanoparticle, a calcium phosphate nanoparticle, etc. The delivery vehicle may or may not comprise a fluid (e.g., a liquid or gas). The delivery vehicle may have various shapes and sizes. For example, the delivery vehicle may be in the shape of a sphere, cuboid, or disc, or any partial shape or combination of shapes thereof. The delivery vehicle may have a cross-section that is circular, triangular, square, rectangular, pentagonal, hexagonal, or any partial shape or combination of shapes thereof.

Examples of the non-viral delivery vehicle may comprise nanoparticles, nanospheres, nanocapsules, microparticies, microspheres, microcapsules, liposomes, nanoemulsions, solid lipid nanoparticles, modifications thereof, or combinations thereof. The non-viral delivery vehicle of the present disclosure may be prepared by methods, such as, but not limited to, nanoprecipitation, emulsion solvent evaporation method, emulsion-crosslinking method, emulsion solvent diffusion method, microemulsion method, gas antisolvent precipitation method, ionic gelation methods milling or size reduction method, PEGylation method, salting-out method, dialysis method, single or double emulsification method, nanospray drying method, layer by layer method, desolvation method, supercritical fluid technology, supramolecular assembly, or combinations thereof.

In some cases, the method can further comprise integrating into the genome of the cell a nucleic acid sequence (e.g., a polynucleotide) encoding at least a portion of the first chimeric polypeptide and/or the second chimeric polypeptide, as provided herein in the present disclosure. In some cases, the nucleic acid sequence may encode at least a portion of the GMP. In some cases, the nucleic acid sequence (e.g., a polynucleotide) encoding the at least the portion of the first and/or second chimeric polypeptides may be integrated into the genome of the cell. Upon administration of the nucleic acid encoding the at least the portion of the first and/or second chimeric polypeptides (e.g., with or without the delivery vehicle), at least a portion of the nucleic acid may be integrated into the genome of the cell. The at least the portion of the integrated nucleic acid may be placed under the control of an autologous promoter of the cell. Alternatively or in addition to, the at least a portion of the integrated nucleic acid may further comprise a promoter that is autologous or heterologous (e.g., a heterologous promoter) to the cell. The heterologous promoter may be configured to bind one or more molecules (e.g., an RNA polymerase, a transcription factor, etc.) that are homologous or heterologous to the cell.

The cell may be in vivo and/or ex vivo (e.g., in vitro) during the treatment with the delivery vehicle comprising a payload (e.g., the at least the portion of the first and/or second chimeric polypeptides, the nucleic acid that encodes the at least the portion of the first and/or second chimeric polypeptides, etc.).

In some cases, the delivery vehicle comprising the payload may be injected into a bodily part of a subject (e.g., a vein, a marrow, etc. of a patient), and the delivery vehicle may interact with (e.g., enter into) the cell in vivo. Other examples of the injection method may include intradermal, subcutaneous, intramuscular, intravenous, intraosseous, intraperitoneal, intrathecal, epidural, intracardiac, intraarticular, intracavernous, and/or intravitreal.

In some cases, the subject may be injected with a dose of the delivery vehicle comprising the payload for at least about 1, 2, 3, 4, 5, 6, 7, 8, 9, 10, or more times. In some cases, the subject may be injected with a dose of the delivery vehicle comprising the payload for at most about 10, 9, 8, 7, 6, 5, 4, 3, 2, or 1 time. In some cases, the subject may be injected with a dose of the delivery vehicle comprising the payload at a frequency of at least once every 1, 2, 3, 4, 5, 6, 7, 8, 9, 10, 20, 30, 60, 90, 180, 360, or more days. In some cases, the subject may be injected with a dose of the delivery vehicle comprising the payload at a frequency of at most once every 360, 180, 90, 60, 30, 20, 10, 9, 8, 7, 6, 5, 4, 3, 2, or 1 day.

In some cases, the cell may be isolated from the subject, and the isolated cell may be treated (e.g., cultured in a culture media) with the delivery vehicle comprising the payload. The isolated cell may be allowed or stimulated to proliferate prior to, during, and/or subsequent to the treatment with the delivery vehicle comprising the payload. In some cases, the cell of interest may be an immune cell. In such a case, the immune cell (e.g., a T cell) may be isolated from the subject. Alternatively or in addition to, a cell that is not the immune cell (e.g., a stem cell, a skin cell, etc.) may be isolated from the subject, and the isolated cell may be induced to differentiate into the immune cell, trans-differentiate into the immune cell, and/or express one or more markers (e.g., one or more TCR complexes) indicative of the immune cell prior to the treatment with the delivery vehicle comprising a payload. In some cases, the cell that is not the immune cell may first be de-differentiated into an induced pluripotent stem cell (iPSC) prior to differentiation into the immune cell (e.g., the T cell) and/or inducing expression of the one or more TCR complexes. Following, the isolated and treated cell may be injected (transplanted) into the subject.

Any of the cells provided herein that are treated (ex vivo and/or in vivo) with at least the payload to administer the GMR comprising the actuator moiety may be referred to as an engineered cell (e.g., an engineered immune cell, such as an engineered T cell).

In some cases, such engineered cell may be injected into a bodily part of a subject (e.g., a vein, a marrow, etc. of a patient), and the delivery vehicle may interact with (e.g., enter into) the cell in vivo. Other examples of the injection method may include intradermal, subcutaneous, intramuscular, intravenous, intraosseous, intraperitoneal.

In some cases, the subject may be injected with a dose of the engineered cells (e.g., cells administered with the GMP) for at least about 1, 2, 3, 4, 5, 6, 7, 8, 9, 10, or more times. In some cases, the subject may be injected with a dose of the engineered cells for at most about 10, 9, 8, 7, 6, 5, 4, 3, 2, or 1 time. In some cases, the subject may be injected with a dose of the engineered cells at a frequency of at least once every 1, 2, 3, 4, 5, 6, 7, 8, 9, 10, 20, 30, 60, 90, 180, 360, or more days. In some cases, the subject may be injected with a dose of the engineered cells at a frequency of at most once every 360, 180, 90, 60, 30, 20, 10, 9, 8, 7, 6, 5, 4, 3, 2, or 1 day.

In some cases, the subject may be injected with at least about 0.5, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 30, 40, 50, 60, 70, 80, 90, 100 (×10⁹) of the engineered cells, or more. In other cases, the subject may be injected with at most about 100, 90, 80, 70, 60, 50, 40, 30, 20, 19, 18, 17, 16, 15, 14, 13, 12, 11, 10, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5 (×10⁹) of the engineered cells, or less.

In some cases, the GMP may be a portion of a chimeric polypeptide. The chimeric polypeptide may or may not be a transmembrane protein. In an example, the chimeric polypeptide may be a CAR, and the GMP may be at least a portion of an intracellular domain of the CAR. In another example, the chimeric polypeptide may be a chimeric transmembrane protein, and the GMP may be at least a portion of an intracellular domain of the chimeric transmembrane protein. In a different example, the chimeric polypeptide comprising the GMP may be an intracellular protein.

In some cases, the administration of the GMP to the cell can comprise treating the cell with at least a portion of the chimeric polypeptide comprising the GMP and/or a polynucleotide that encodes the at least a portion of the chimeric polypeptide comprising the GMP. Such treatment may occur in the presence or absence of one or more delivery vehicles provided herein in the present disclosure. In some cases, the method can further comprise administering to the cell a chimeric polypeptide comprising the GMP, wherein the chimeric polypeptide is operable to release the GMP from the chimeric polypeptide in response to a stimulant (e.g., the ligand of the receptor provided herein in the present disclosure), and wherein the released GMP is operable to regulate expression of the target polynucleotide in the cell. In some cases, the method can further comprise administering to the cell a chimeric polypeptide comprising the GMP and a nuclear localization domain, wherein the nuclear localization domain is operable to translocate the chimeric polypeptide to a nucleus of the cell in response to a stimulant, and wherein the translocated GMP is operable to regulate expression of the target polynucleotide in the cell.

In some cases, the nuclear localization domain can be derived from a transcription factor, as abovementioned. The transcription factor can be a regulatable transcription factor that is only active and able to translocate into a nucleus in response to a signal or signaling pathway. The transcription factor can be a regulatable transcription factor that is primarily active and able to translocate into a nucleus in response to a signal or signaling pathway. The transcription factor can be a regulatable transcription factor that is generally active and able to translocate into a nucleus in response to a signal or signaling pathway.

In some examples, the nuclear localization domain can be derived from the NFAT family members (e.g., NFATp, NFAT1, NFATc1, NFATc2, NFATc3, NFAT4, NFATx, NFATc4, NFAT3, and NFAT5), nuclear factor kappa B (NF-κB), NFKB1 p50, activator protein 1 (AP-1), signal transducer and activator of transcription family members (e.g., STAT1, STAT2, STAT3, STAT4, STAT5A, STAT5B, and STATE), sterol response element-binding proteins (e.g., SREBP-1 and SREBF1), a light or circadian or electromagnetic sensing protein such as cryptochromes (e.g., CRY1, CRY2), Timeless (TIM), PAS domain of PER proteins (e.g., PER1, PER2, and PER3), or other transcription factors or signal transducers.

In some cases, upon activation of the receptor of the cell (e.g., an endogenous receptor, such as TCR of an immune cell), the actuator moiety of the GMP can be activated to regulate expression of the target polynucleotide in the cell (e.g., endogenous gene of the cell) by at least a 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a control cell. In some cases, upon activation of the receptor of the cell (e.g., an endogenous receptor, such as TCR of an immune cell), the actuator moiety of the GMP GMP can be activated to regulate expression of the target polynucleotide in the cell (e.g., endogenous gene of the cell) by at most 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a control cell. In some cases, upon activation of the receptor of the cell (e.g., an endogenous receptor, such as TCR of an immune cell), the actuator moiety of the GMP can be activated to regulate expression of the target polynucleotide in the cell (e.g., endogenous gene of the cell) by at least about 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 100%, 110%, 120%, 130%, 140%, 150%, 200%, or more as compared to a control cell. In some cases, upon activation of the receptor of the cell (e.g., an endogenous receptor, such as TCR of an immune cell), the actuator moiety of the GMP can be activated to regulate expression of the target polynucleotide in the cell (e.g., endogenous gene of the cell) by at most about 200%, 150%, 140%, 130%, 120%, 110%, 100%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, 0.5%, 0.1%, or less as compared to a control cell.

A control cell as disclosed herein be a cell that (i) lacks (or exhibits a lower expression level of) a first chimeric polypeptide comprising the GMP and (ii) lacks (or exhibits a lower expression level of) a second chimeric polypeptide comprising the cleavage moiety capable of releasing (and activating) the actuator moiety from the GMP. Alternatively, a control cell can be a cell that (i) comprises a first chimeric polypeptide comprising the GMP and (ii) lacks (or exhibits a lower expression level of) a second chimeric polypeptide comprising the cleavage moiety capable of releasing (and activating) the actuator moiety from the GMP. Alternatively, a control cell can be a cell that (i) lacks (or exhibits a lower expression level of) a first chimeric polypeptide comprising the GMP and (ii) comprises a second chimeric polypeptide comprising the cleavage moiety capable of releasing (and activating) the actuator moiety from the GMP. Alternatively, a control cell can be a cell that lacks (or exhibits a lower expression level of) a guide nucleic acid (e.g., sgRNA) capable of binding the target polynucleotide. Alternatively, a control cell can be a cell that comprises a control guide nucleic acid (e.g., sgRNA) that is not capable of binding the target polynucleotide.

A target polynucleotide as disclosed herein can be a DNA molecule (e.g., genomic or non-genomic DNA sequence). Alternatively, the target polynucleotide can be an RNA molecule (e.g. mRNA). Expression level of the target polynucleotide can be enhanced by action of the actuator moiety. Alternatively, expression level of the target polynucleotide can be reduced by action of the actuator moiety. The target polynucleotide can be involved in (e.g., directly involved in) cell regulation (e.g., immune cell regulation). The target polynucleotide can encode a protein that is involved in cell regulation (e.g., immune cell regulation). Such cell regulation can enhance activity of the cell (e.g., activity of the immune cell) or reduce activity of the cell. In some cases, the target polynucleotide can encode an immune checkpoint regulator (e.g., an immune checkpoint inhibitor, such as A2AR, B7-H3, B7-H4, BTLA, CTLA-4, IDO, KIR, LAG3, NOX2, PD-1, TIM-3, VISTA, SIGLC7, etc.). In some cases, the target polynucleotide can encode a cytokine of the cell. The cytokine can comprise interleukin (IL) selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. For example, the target polynucleotide can encode IL-12 (e.g., IL12A and/or IL12B). In some cases, the target polynucleotide can encode a cell fate control gene. Non-limiting examples of cell fate control genes can include: Pax (e.g., PAX-1, PAX-2, PAX-3, PAX-4, PAX-5, PAX-6, PAX-7, PAX-8 or PAX-9); HOX (e.g., HOX A1-7, 9-11 or 13; HOX B1-9; HOX C4-6 or 8-13; HOX DE 3-4 or 8-13), DLX (e.g., DLX-2, DLX-4, DLX-5; mouse DLX-1, DLX-2, DLX-3, DLX-5, DLX-6; DLX-7;), PBC (e.g., Pbx1, Pbx2 or Pbx3), MEINOX (e.g., Meis1, Meis2, Meis3), bHLH (e.g., MyoD, myogenin, myf-5, MASH-1 and MASH-2), LEVI homeobox (e.g., ISLET-1, LEVI-1, LMX1B, LHX2), MSX (e.g., MSX-1, or MSX-2), POU (e.g., Oct-1, Oct-2, Oct-6 and Pit-1), PTX (e.g., Ptx1, Ptx2), NKX (e.g., NKX2.5, NKX2.8, NKX3.1), MADS box (e.g., SRF and mef2 and), SOX (e.g., SOX-2, SOX-4, SOX-8, SOX-9, SOX-10, SOX-11, SOX-14 and SOX-17), T-box (e.g., TBX-5, TBX-6, TBX-10, TBX-18, TBX-19, TBX-20, TBX-21), WNT (e.g., WNT-1, WNT-2, WNT-3A, WNT4, WNT-5A, WNT-7a, WNT-7B, WNT-8A, WNT-10B, WNT-13, WNT-14), BMP/TGF (e.g., TGF(31, TGF(32, TGF(33, BMP-1, BMP-2, BMP-3B (GDF10), BMP-4, BMP-5, BMP-6, BMP-7, BMP-8, activin, GDF1, GDF5, GDF8, GDF9), and hedgehog (e.g., Sonic, Indian or Desert hedgehog). Alternatively or in addition to, the target polynucleotide can encode a respective receptor of any of the cytokine or cell fate control gene. Non-limiting examples of such receptor can include IL receptor, WNT receptor, BMP receptor, TGF receptor (e.g., TGFbeta receptor, such as TGFbeta receptor 2), hedgehog receptor (e.g., PTCH1, PTCH2), etc. For example, the actuator moiety of the GMP, upon activation thereof, can downregulate expression or activity of TGF receptor (e.g., TGFbeta receptor 2).

In some cases, upon activation of the receptor of the cell, the actuator moiety of the GMP can be activated to upregulate expression or activity of a target polynucleotide in the cell. In some examples, upregulated expression or activity of the target polynucleotide in the cell can improve cell function, such as cell fitness (e.g., T cell fitness), stemness, prevention of exhaustion, survival, and/or expansion. Non-limiting examples of target polynucleotides that can be upregulated by the systems and methods disclosed herein can include Id1/Id3 (e.g., for improving or prolonging stemness), cJun or Jun (e.g., for prevention of T cell exhaustion), TBX (e.g., TBX-21 for improving or prolonging stemness), and interleukin (e.g., IL-21 for improving survival or expansion of the cells).

In some cases, upon activation of the receptor of the cell, the actuator moiety of the GMP can be activated to downregulate expression or activity of a target polynucleotide in the cell. In some examples, downregulated expression or activity of the target polynucleotide in the cell can enhance cell potency and/or survivability (e.g., T cell potency and/or survivability in tumors). Non-limiting examples of target polynucleotides that can be downregulated by the systems and methods disclosed herein can include TOX (e.g., TOX2 for reducing or preventing T cell exhaustion), SOCS (e.g., SOCS-1 for enhancing T cell potency against a target cell, such as a cancer or diseased cell), SHIP (e.g., SHIP-1 for reducing or preventing T cell exhaustion), Basic Leucine Zipper ATF-Like Transcription Factor (BATF) (e.g., for reducing or preventing T cell exhaustion), and Beta-2-Microglobulin (B2M) (e.g., for enhancing immunogenicity against a target cell, such as a cancer or diseased cell).

In some cases, the GMP can regulate expression or activity of the target polynucleotide in the cell for at least 1 minute, 5 minutes, 10 minutes, 30 minutes, 1 hour, 2 hours, 3 hours, 4 hours, 5 hours, 6 hours, 7 hours, 8 hours, 12 hours, 16 hours, 20 hours, 24 hours, 2 days, 3 days, 4 days, 5 days, 6 days, 7 days, 2 weeks, 3 weeks, 4 weeks, 2 months, 4 months, 6 months, 1 year, or more in comparison to the cell in the absence of the GMP. In some cases, the GMP can regulate expression or activity of the target polynucleotide in the cell for at most 1 year, 6 months, 4 months, 2 months, 4 weeks, 3 weeks, 2 weeks, 7 days, 6 days, 5 days, 4 days, 3 days, 2 days, 24 hours, 20 hours, 16 hours, 12 hours, 8 hours, 7 hours, 6 hours, 5 hours, 4 hours, 3 hours, 2 hours, 1 hour, 30 minutes, 10 minutes, 5 minutes, 1 minute, or less in comparison to the cell in the absence of the GMP.

The regulating the expression or activity of the target polynucleotide in the cell can comprise decreasing, increasing, inhibiting, and/or prolonging the expression or activity of the target polynucleotide in the cell. The regulating the expression or activity of the target polynucleotide in the cell can be decreasing the expression or activity of the target polynucleotide in the cell. The regulating the expression or activity of the target polynucleotide in the cell can be increasing the expression or activity of the target polynucleotide in the cell.

The regulating the expression or activity of the target polynucleotide in the cell may directly and/or indirectly allow the regulating the activity of the cell. In some cases, the regulating the activity of the cell can comprise decreasing and/or inhibiting self-inflicted injury of the cell, death of the cell by another cell, and/or death of another cell by the cell, thereby improving (directly and/or indirectly) viability, proliferation, and/or function of the cell.

In some cases, the regulating the activity of the cell can comprise inducing and/or prolonging activation of the cell (e.g., activation of the immune cell, such as the T cell). The activation of the cell can comprise activation of one or more biological activities (e.g., migration, proliferation, synthesis of one or more polypeptides, etc.) of the cell.

In some cases, the GMP may be configured to reduce and/or prevent activation of the cell.

In some cases, the GMP comprising the actuator moiety may be configured to increase or decrease expression or activity of one or more angiogenic factors in the cell. In some cases, the GMP comprising the actuator moiety may be configured to decrease expression or activity of one or more angiogenic factors in the cell. In some cases, the GMP comprising the actuator moiety may be configured to decrease expression or activity of one or more angiogenic factors in the cell. The GMP comprising the actuator moiety may be expressed along with a guide RNA (e.g., sgRNA) against one or more polynucleotide sequences encoding for the one or more angiogenic factors in the T cell. The actuator moiety of the GMP, in conjunction with the guide RNA, may be configured to increase or decrease expression or activity of one or more angiogenic factors in the cell.

The one or more angiogenic factors can include pro-angiogenic factors and/or anti-angiogenic factors. Examples of the pro-angiogenic factors can include, but are not limited to, FGF, VEGF, VEGFR, NRP-1, Ang1, Ang2, PDGF (BB-homodimer), PDGFR, TGF-β, endoglin, TGF-βreceptors, MCP-1, Integrins α_{ν}β₃, α_{ν}β₃, α₅β₁, VE-Cadherin, CD31, ephrin, plasminogen activators, plasminogen activator inhibitor-1, eNOS, COX-2, AC133, Id1/Id3, Angiogenin, HGF, Vegf, IL-17, IL-1 alpha, IL-8, IL-6, Cxcl5, Fgfα, Fgfβ, Tgfα, Tgfβ, MMPs (including mmp9), Plasminogen activator inhibitor-1, Thrombospondin, Angiopoietin 1, Angiopoietin 2, Amphiregulin, Leptin, Endothelin-1, AAMP, AGGF1, AMOT, ANGLPTL3, ANGPTL4, BTG1, IL-1βNOS3, TNFSF12, and/or VASH2.

In some cases, a nucleic acid sequence encoding the GMP may be integrated into a genome of the cell.

In some cases, the cleavage recognition site may comprise a polypeptide sequence, and the cleavage moiety may comprise protease activity. In some cases, the cleavage recognition site may comprise a disulfide bond, and the cleavage moiety may comprise oxidoreductase activity. In some cases, the cleavage recognition site may comprise a first portion of an intein sequence that reacts with a second portion of the intein sequence to release the actuator moiety.

In some cases, the cleavage moiety can only cleave the recognition site when in proximity to the cleavage recognition site. The cleavage recognition site can comprise a polypeptide sequence that is a recognition sequence of a protease. The cleavage moiety can comprise protease activity which recognizes the polypeptide sequence. A cleavage moiety comprising protease activity can be a protease, or any derivative, variant or fragment thereof. A protease can refer to any enzyme that performs proteolysis, in which polypeptides are cleaved into smaller polypeptides or amino acids. Various proteases can be suitable for use as a cleavage moiety. Some proteases can be highly promiscuous such that a wide range of protein substrates are hydrolysed. Some proteases can be highly specific and only cleave substrates with a certain sequence, e.g., a cleavage recognition sequence or peptide cleavage domain. In some cases, the cleavage recognitions site can comprise multiple cleavage recognition sequences, and each cleavage recognition sequence can be recognized by the same or different cleavage moiety comprising protease activity (e.g., protease). Sequence-specific proteases that can be used as cleavage moieties include, but are not limited to, superfamily CA proteases, e.g., families C1, C2, C6, C10, C12, C16, C19, C28, C31, C32, C33, C39, C47, C51, C54, C58, C64, C65, C66, C67, C70, C71, C76, C78, C83, C85, C86, C87, C93, C96, C98, and C101, including papain (Carica papaya), bromelain (Ananas comosus), cathepsin K (liverwort) and calpain (Homo sapiens); superfamily CD proteases, e.g., family C11, C13, C14, C25, C50, C80, and C84: such as caspase-1 (Rattus norvegicus) and separase (Saccharomyces cerevisiae); superfamily CE protease, e.g., family C5, C48, C55, C57, C63, and C79 including adenain (human adenovirus type 2); superfamily CF proteases, e.g., family C15 including pyroglutamyl-peptidase I (Bacillus amyloliquefaciens); superfamily CL proteases, e.g., family C60 and C82 including sortase A (Staphylococcus aureus); superfamily CM proteases, e.g. family C18 including hepatitis C virus peptidase 2 (hepatitis C virus); superfamily CN proteases, e.g., family C9 including sindbis virus-type nsP2 peptidase (sindbis virus); superfamily CO proteases, e.g., family C40 including dipeptidyl-peptidase VI (Lysinibacillus sphaericus); superfamily CP proteases, e.g., family C97 including DeSI-1 peptidase (Mus musculus); superfamily PA proteases, e.g., family C3, C4, C24, C30, C37, C62, C74, and C99 including TEV protease (Tobacco etch virus); superfamily PB proteases, e.g., family C44, C45, C59, C69, C89, and C95 including amidophosphoribosyltransferase precursor (Homo sapiens); superfamily PC proteases, families C26, and C56 including Û-glutamyl hydrolase (Rattus norvegicus); superfamily PD proteases, e.g., family C46 including Hedgehog protein (Drosophila melanogaster); superfamily PE proteases, e.g., family P1 including DmpA aminopeptidase (Ochrobactrum anthropi); others proteases, e.g., family C7, C8, C21, C23, C27, C36, C42, C53 and C75. Additional proteases include serine proteases, e.g., those of superfamily SB, e.g., families S8 and S53 including subtilisin (Bacillus licheniformis); those of superfamily SC, e.g., families S9, S10, S15, S28, S33, and S37 including prolyl oligopeptidase (Sus scrofa); those of superfamily SE, e.g., families S11, S12, and S13 including D-Ala-D-Ala peptidase C (Escherichia coli); those of superfamily SF, e.g., families S24 and S26 including signal peptidase I (Escherichia coli); those of Superfamily SJ, e.g., families S16, S50, and S69 including lon-A peptidase (Escherichia coli); those of Superfamily SK, e.g., families S14, S41, and S49 including Clp protease (Escherichia coli); those of Superfamily SO, e.g., families S74 including Phage K1F endosialidase CIMCD self-cleaving protein (Enterobacteria phage K1F); those of superfamily SP, e.g., family S59 including nucleoporin 145 (Homo sapiens); those of superfamily SR, e.g., family S60 including Lactoferrin (Homo sapiens); those of superfamily SS, families S66 including murein tetrapeptidase LD-carboxypeptidase (Pseudomonas aeruginosa); those of superfamily ST, e.g., families S54 including rhomboid-1 (Drosophila melanogaster); those of superfamily PA, e.g., families S1, S3, S6, S7, S29, S30, S31, S32, S39, S46, S55, S64, S65, and S75 including Chymotrypsin A (Bos taurus); those of superfamily PB, e.g., families S45 and S63 including penicillin G acylase precursor (Escherichia coli); those of superfamily PC, e.g., families S51 including dipeptidase E (Escherichia coli); those of superfamily PE, e.g., families P1 including DmpA aminopeptidase (Ochrobactrum anthropi); those unassigned, e.g., families S48, S62, S68, S71, S72, S79, and S81 threonine proteases, e.g., those of superfamily PB clan, e.g., families T1, T2, T3, and T6 including archaean proteasome, űcomponent (Thermoplasma acidophilum); and those of superfamily PE clan, e.g., family T5 including ornithine acetyltransferase (Saccharomyces cerevisiae); aspartic proteases, e.g., BACE1, BACE2; cathepsin D; cathepsin E; chymosin; napsin-A; nepenthesin; pepsin; plasmepsin; presenilin; renin; and HIV-1 protease, and metalloproteinases, e.g., exopeptidases, metalloexopeptidases; endopeptidases, and metalloendopeptidases. A cleavage recognition sequence (e.g., polypeptide sequence) can be recognized by any of the proteases disclosed herein.

In some cases, the cleavage recognition site can comprise a cleavage recognition sequence (e.g., polypeptide sequence or peptide cleavage domain) that is recognized by a protease selected from the group consisting of: achromopeptidase, aminopeptidase, ancrod, angiotensin converting enzyme, bromelain, calpain, calpain I, calpain II, carboxypeptidase A, carboxypeptidase B, carboxypeptidase G, carboxypeptidase P, carboxypeptidase W, carboxypeptidase Y, caspase 1, caspase 2, caspase 3, caspase 4, caspase 5, caspase 6, caspase 7, caspase 8, caspase 9, caspase 10, caspase 11, caspase 12, caspase 13, cathepsin B, cathepsin C, cathepsin D, cathepsin E, cathepsin G, cathepsin H, cathepsin L, chymopapain, chymase, chymotrypsin, clostripain, collagenase, complement C1r, complement C1s, complement Factor D, complement factor I, cucumisin, dipeptidyl peptidase IV, elastase (leukocyte), elastase (pancreatic), endoproteinase Arg-C, endoproteinase Asp-N, endoproteinase Glu-C, endoproteinase Lys-C, enterokinase, factor Xa, ficin, furin, granzyme A, granzyme B, HIV Protease, IGase, kallikrein tissue, leucine aminopeptidase (general), leucine aminopeptidase (cytosol), leucine aminopeptidase (microsomal), matrix metalloprotease, methionine, aminopeptidase, neutrase, papain, pepsin, plasmin, prolidase, pronase E, prostate specific antigen, protease alkalophilic from Streptomyces griseus, protease from Aspergillus, protease from Aspergillus saitoi, protease from Aspergillus sojae, protease (B. licheniformis) (alkaline or alcalase), protease from Bacillus polymyxa, protease from Bacillus sp, protease from Rhizopus sp., protease S, proteasomes, proteinase from Aspergillus oryzae, proteinase 3, proteinase A, proteinase K, protein C, pyroglutamate aminopeptidase, rennin, rennin, streptokinase, subtilisin, thermolysin, thrombin, tissue plasminogen activator, trypsin, tryptase and urokinase.

Further details of proteases and associated recognition sequences that can be used in systems and methods of the present disclosure are disclosed in Patent Cooperation Treaty (PCT) Patent Application No. PCT/US17/012885 and PCT Patent Application No. PCT/US17/012881, each of which is incorporated in its entirety herein by reference.

In some cases, the actuator moiety of the GMP can be an RNA-guided actuator moiety or a variant thereof, which RNA-guided actuator moiety forms a complex with the target polynucleotide. In some cases, the actuator moiety can be a CRISPR-associated (Cas) protein or a fragment thereof that substantially lacks DNA cleavage activity. In some cases, the actuator moiety can be Cas9 and/or Cpf1. In some cases, the actuator moiety can comprise an activator effective to increase expression or activity of the target polynucleotide. In some cases, the actuator moiety can comprise a repressor effective to decrease expression or activity of the target polynucleotide.

Further details of design and application of systems comprising the chimeric polypeptide (e.g., chimeric receptor polypeptide, the chimeric adaptor polypeptide, etc.), CAR, GMP, ligands (e.g., antigens), modifications thereof, and expression cassettes comprising thereof are disclosed in PCT Patent Application No. PCT/US17/012885, PCT Patent Application No. PCT/US17/012881, PCT Patent Application No. PCT/US18/041704, U.S. Pat. No. 9,856,497, U.S. Non-Provisional application Ser. No. 15/806,756, U.S. Non-Provisional application Ser. No. 16/029,299, U.S. Non-Provisional application Ser. No. 16/029,299, U.S. Provisional Application No. 62/639,427, U.S. Provisional Application No. 62/639,386, U.S. Provisional Application No. 62/647,543, and U.S. Provisional Application No. 62/675,134, each of which is incorporated in its entirety herein by reference.

Contacting the cell with the ligand can occur directly and/or indirectly. Direct stimulation may occur when the ligand binds a portion of the cell. In some cases, the ligand may bind to the receptor of the cell. In an example, the ligand may bind to a ligand binding domain of the receptor. Indirect stimulation can occur when the ligand activates or deactivates a different cell, which different cell is operable to activate the cell by using its cell surface marker (e.g., a cell surface ligand) to bind the receptor of the cell. Consequentially, the cell may be activated to regulate expression or activity of the target polynucleotide in the cell. The different cell may be of the same (e.g., another cell of the same type) or different cell type than the cell.

Contacting the cell with the ligand may occur prior to, during, and/or subsequent to administration of the GMP comprising the actuator moiety to the cell. The cell may be ex vivo and/or in vivo during the contacting of the cell (e.g., the receptor of the cell) with the ligand.

Contacting the cell with the ligand may occur prior to, during, and/or subsequent to administration of the cell (e.g., the engineered cell) to a subject. The cell may be contacted with the ligand prior to, during, and/or subsequent to administration of the cell to the subject for a duration of time of at least about 0.5, 1, 1.5, 2, 2.5, 3, 3.5, 4, 4.5, 5, 5.5, 6, 6.5 days, or more. The cell may be contacted with the ligand prior to, during, and/or subsequent to administration of the cell to the subject fora duration of time of at most about 6.5, 6, 5.5, 5, 4.5, 4, 3.5, 3, 2.5, 2, 1.5, 1, 0.5 days, or less. The cell may be contacted with the ligand for a duration of time of at least about 0.5, 1, 1.5, 2, 2.5, 3, 3.5, 4, 4.5, 5, 5.5, 6, 6.5 days, or more prior to administration of the cell to the subject. The cell may be contacted with the ligand for a duration of time of at most about 6.5, 6, 5.5, 5, 4.5, 4, 3.5, 3, 2.5, 2, 1.5, 1, 0.5 days, or less prior to administration of the cell to the subject. The cell may be contacted with the ligand for a duration of time of at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 days, or more subsequent to administration of the cell to the subject. The cell may be contacted with the ligand for a duration of time of at most about 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10 days, or less subsequent to administration of the cell to the subject. In some cases, the cell may be contacted with the ligand at least about 1, 2, 3, 4, 5, 6, 7, 8, 9, 10 times, or more. In some cases, the cell may be contacted with the ligand at most about 10, 9, 8, 7, 6, 5, 4, 3, 2, or 1 time. In some cases, the cell may be contacted with the ligand at a dose concentration of at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 900, 1000 international units per millilitre (IU/mL), or more. In other cases, the cell may be contacted with the ligand at a dose concentration of at most about 1000, 900, 800, 790, 780, 770, 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10 IU/mL, or less.

In some cases, the ligand (i.e., the stimulant) of the receptor of the cell may be selected from the group consisting of interleukins (e.g., IL-2), interferons, transforming growth factors (TGFs), ligands for cluster of differentiation (CD) receptors, and variants thereof. The stimulant may be an antigen described in the subject disclosure. In some examples, the antigen may induce migration, survival, proliferation, and/or differentiation of an immune cell (e.g., a T cell). In some cases, the stimulant may comprise a vaccine (e.g., an immune cell vaccine). A vaccine may be a pharmaceutical composition comprising at least one immunologically protective molecule that induces an immunological and/or protective response in a cell (e.g., an immune cell) or an animal. A vaccine may further comprise one or more additional components (e.g., adjuvants) that enhance the immunological activity. In an example, the immune cell vaccine may be a peptide vaccine (e.g., p-27L) or a viral vaccine (e.g., p-210M, rFP-210M).

In some cases, the ligand binding domain (e.g., the stimulant binding domain) of the cell binds an antigen that is not membrane bound (e.g., non-membrane-bound), for example an extracellular antigen that is secreted by a cell (e.g., a target cell) or an antigen located in the cytoplasm of a cell (e.g., a target cell). Antigens (e.g., membrane bound and non-membrane bound) can be associated with a disease such as a viral, bacterial, and/or parasitic infection; inflammatory and/or autoimmune disease; or neoplasm such as a cancer and/or tumor. Non-limiting examples of antigens which can be bound by a ligand binding domain of a chimeric transmembrane receptor polypeptide of a subject system include, but are not limited to, 1-40-β-amyloid, 4-1BB, SAC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β3 (ADRB3), AGS-22M6, α olate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (α chain of IL-2 receptor), CD27, CD274, CD28, CD3, CD3 ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C—X—C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRL5), fetal acetylcholine receptor, fibrin II (3 chain, fibroblast activation protein a (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/βreceptor, interferon γ-induced protein, Interleukin 11 receptor α(IL-11Ra), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIRD, Lewis-Y antigen, LFA-1 (CD11 a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-R a), platelet-derived growth factor receptor β (PDGFR-β), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MARTI), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, βType, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, surviving, syndecan 1 (SDC1)+A314, SOX10, survivin, surviving-2B, synovial sarcoma, X breakpoint 2 (SSX2), T-cell receptor, TCR F Alternate Reading Frame Protein (TARP), telomerase, TEM1, tenascin C, TGF-β(e.g., TGF-β 1, TGF-β 2, TGF-β 3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, and κlight chain, and variants thereof.

In some embodiments, the ligand binding domain binds an antigen selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (ela2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2M, HST-2 (FGF6), HST-2/neu, hTERT, iCE, IL-11Ra, IL-13Ra2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1Nlelan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, 0A1, OGT, oncofetal antigen (h5T4), OS-9, P polypeptide, P15, P53, PRAIVIE, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain. In some embodiments, the ligand binding domain binds to a tumor associated antigen.

In some embodiments, the target polynucleotide encodes for a cytokine. Non-limiting examples of cytokines include 4-1BBL, activin βA, activin βB, activin βC, activin βD, artemin (ARTN), BAFF/BLyS/TNFSF138, BMP10, BMP15, BMP2, BMP3, BMP4, BMP5, BMP6, BMP7, BMP8a, BMP8b, bone morphogenetic protein 1 (BMP1), CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CD153/CD30L/TNFSF8, CD40L/CD154/TNFSF5, CD40LG, CD70, CD70/CD27L/TNFSF7, CLCF1, c-MPL/CD110/TPOR, CNTF, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, EDA-A1, FAM19A1, FAM19A2, FAM19A3, FAM19A4, FAM19A5, Fas Ligand/FASLG/CD95L/CD178, GDF10, GDF11, GDF15, GDF2, GDF3, GDF4, GDF5, GDF6, GDF7, GDF8, GDF9, glial cell line-derived neurotrophic factor (GDNF), growth differentiation factor 1 (GDF1), IFNA1, IFNA10, IFNA13, IFNA14, IFNA2, IFNA4, IFNA5/IFNaG, IFNA7, IFNA8, IFNB1, IFNE, IFNG, IFNZ, IFNω/IFNW1, IL11, IL18, IL18BP, ILIA, IL1B, IL1F10, IL1F3/IL1RA, IL1F5, IL1F6, IL1F7, IL1F8, IL1F9, IL1RL2, IL31, IL33, IL6, IL8/CXCL8, inhibin-A, inhibin-B, Leptin, LIF, LTA/TNFB/TNFSF1, LTB/TNFC, neurturin (NRTN), OSM, OX-40L/TNFSF4/CD252, persephin (PSPN), RANKL/OPGL/TNFSF11(CD254), TL1A/TNFSF15, TNFA, TNF-alpha/TNFA, TNFSF10/TRAIL/AP0-2L(CD253), TNFSF12, TNFSF13, TNFSF14/LIGHT/CD258, XCL1, and XCL2. In some embodiments, the target gene encodes for an immune checkpoint inhibitor. Non-limiting examples of such immune checkpoint inhibitors include PD-1, CTLA-4, LAG3, TIM-3, A2AR, B7-H3, B7-H4, BTLA, DO, KIR, and VISTA. In some embodiments, the target gene encodes for a T cell receptor (TCR) alpha, beta, gamma, and/or delta chain.

A subject system can be introduced in a variety of immune cells, including any cell that is involved in an immune response. In some embodiments, immune cells comprise granulocytes such as asophils, eosinophils, and neutrophils; mast cells; monocytes which can develop into macrophages; antigen-presenting cells such as dendritic cells; and lymphocytes such as natural killer cells (NK cells), B cells, and T cells. In some embodiments, an immune cell is an immune effector cell. An immune effector cell refers to an immune cell that can perform a specific function in response to a stimulus. In some embodiments, an immune cell is an immune effector cell which can induce cell death. In some embodiments, the immune cell is a lymphocyte. In some embodiments, the lymphocyte is a NK cell. In some embodiments the lymphocyte is a T cell. In some embodiments, the T cell is an activated T cell. T cells include both naive and memory cells (e.g. central memory or T_{cm}, effector memory or T_{EM} and effector memory RA or T_{EMRA}), effector cells (e.g. cytotoxic T cells or CTLs or Tc cells), helper cells (e.g. Th1, Th2, Th3, Th9, Th7, TFH), regulatory cells (e.g. Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage. T cells can be divided into two broad categories: CD8+ T cells and CD4+ T cells, based on which protein is present on the cell's surface. T cells expressing a subject system can carry out multiple functions, including killing infected cells and activating or recruiting other immune cells. CD8+ T cells are referred to as cytotoxic T cells or cytotoxic T lymphocytes (CTLs). CTLs expressing a subject system can be involved in recognizing and removing virus-infected cells and cancer cells. CTLs have specialized compartments, or granules, containing cytotoxins that cause apoptosis, e.g., programmed cell death. CD4+ T cells can be subdivided into four sub-sets—Th1, Th2, Th17, and Treg, with “Th” referring to “T helper cell,” although additional sub-sets may exist. Th1 cells can coordinate immune responses against intracellular microbes, especially bacteria. They can produce and secrete molecules that alert and activate other immune cells, like bacteria-ingesting macrophages. Th2 cells are involved in coordinating immune responses against extracellular pathogens, like helminths (parasitic worms), by alerting B cells, granulocytes, and mast cells. Th17 cells can produce interleukin 17 (IL-17), a signaling molecule that activates immune and non-immune cells. Th17 cells are important for recruiting neutrophils.

A variety of cells can be used as a host cell to realize the systems and methods of the subject disclosure. A host cell to which any of the embodiments (e.g., a cell comprising or expressing the γδ TCR complex) disclosed herein can be applied (e.g., transduced) includes a wide variety of cell types. A host cell can be in vitro. A host cell can be in vivo. A host cell can be ex vivo. A host cell can be an isolated cell. A host cell can be a cell inside of an organism. A host cell can be an organism. A host cell can be a cell in a cell culture. A host cell can be one of a collection of cells. A host cell can be a mammalian cell or derived from a mammalian cell. A host cell can be a rodent cell or derived from a rodent cell. A host cell can be a human cell or derived from a human cell. A host cell can be a prokaryotic cell or derived from a prokaryotic cell. A host cell can be a bacterial cell or can be derived from a bacterial cell. A host cell can be an archaeal cell or derived from an archaeal cell. A host cell can be a eukaryotic cell or derived from a eukaryotic cell. A host cell can be a pluripotent stem cell. A host cell can be a plant cell or derived from a plant cell. A host cell can be an animal cell or derived from an animal cell. A host cell can be an invertebrate cell or derived from an invertebrate cell. A host cell can be a vertebrate cell or derived from a vertebrate cell. A host cell can be a microbe cell or derived from a microbe cell. A host cell can be a fungi cell or derived from a fungi cell. A host cell can be from a specific organ or tissue.

A host cell can be an immune cell, as abovementioned in the subject disclosure.

A host cell can be a stem cell or progenitor cell. Host cells can include stem cells (e.g., adult stem cells, embryonic stem cells, induced pluripotent stem (iPS) cells) and progenitor cells (e.g., cardiac progenitor cells, neural progenitor cells, etc.). Host cells can include mammalian stem cells and progenitor cells, including rodent stem cells, rodent progenitor cells, human stem cells, human progenitor cells, etc. Clonal cells can comprise the progeny of a cell. A host cell can be in a living organism. A host cell can be a genetically modified cell.

A host cell can be a totipotent stem cell, however, in some embodiments of this disclosure, the term “cell” may be used but may not refer to a totipotent stem cell. A host cell can be a plant cell, but in some embodiments of this disclosure, the term “cell” may be used but may not refer to a plant cell. A host cell can be a pluripotent cell. For example, a host cell can be a pluripotent hematopoietic cell that can differentiate into other cells in the hematopoietic cell lineage but may not be able to differentiate into any other non-hematopoietic cell. A host cell may be able to develop into a whole organism. A host cell may or may not be able to develop into a whole organism. A host cell may be a whole organism.

A variety of one or more intrinsic signaling pathways (e.g. NFkB) of a cell are available for embodiments provided herein. Table 1 provides exemplary signaling pathways and genes associated with the signaling pathway. A signaling pathway activated by stimulant binding to a cell (e.g., an immune cell, a stem cell, etc.) and/or a ligand binding to a transmembrane receptor in embodiments provided herein can be any one of those provided in Table 1. In an example, a promoter activated to drive expression of the GMP upon binding of a stimulant to the stimulant binding domain of a transmembrane receptor in embodiments provided can comprise the promoter sequence driving any of the genes provided in Table 1, any variant of the promoter sequence, or any partial promoter sequence (e.g., a minimal promoter sequence).

TABLE 1

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; |
| Signaling | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor | PRKCE; ITGAM; ROCK1; ITGA5; CXCR4; IRAK1; |
| Signaling | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; |
| Signaling | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1 GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor | RAC1; PTEN; LYN; ELK1; MAPK1; RAC2; PTPN11; |
| Signaling | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte | ACTN4; CD44; PRKCE; ITGAM; ROCK1; CXCR4; CYBA; |
| Extravasation | RAC1; RAP1A; PRKCZ; ROCK2; RAC2; PTPN11; |
| Signaling | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon | HSPB1; EP300; FASN; TGM2; RXRA; MAPK1; NQO1; |
| Receptor Signaling | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin | CD44; EP300; LRP6; DVL3; CSNK1E; GJA1; SMO; |
| Signaling | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated | PRKCE; EP300; SOD2; PRKCZ; MAPK1; SQSTM1; |
| Oxidative | NQO1; PIK3CA; PRKCI; FOS; PIK3CB; PIK3C3; MAPK8; |
| Stress Response | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell | PRKCE; RAC1; PRKCZ; MAPK1; RAC2; PTPN11; |
| Signaling | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor | RAC1; ELK1; MAPK1; IKBKB; CBL; PIK3CA; FOS; |
| Signaling | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor | CRADD; HSPB1; BID; BIRC4; TBK1; IKBKB; FADD; |
| Signaling | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; |
| Nicotinamide | PLK1; AKT2; CDK8; MAPK8; MAPK3; PRKCD; PRKAA1; |
| Metabolism | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor | IRAK1; EIF2AK2; MYD88; TRAF6; PPARA; ELK1; |
| Signaling | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK | NTRK2; MAPK1; PTPN11; PIK3CA; CREB1; FOS; |
| Signaling | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated | IRAK1; MYD88; TRAF6; PPARA; RXRA; ABCA1; |
| Inhibition of | MAPK8; ALDH1A1; GSTP1; MAPK9; ABCB1; TRAF2; |
| RXR Function | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in | KDR; FLT1; PGF; AKT2; PIK3CA; PIK3CB; PIK3C3; |
| the Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated | RAP1A; MAPK1; GNAS; CREB1; CAMK2A; MAPK3; |
| Signaling | SRC; RAF1; MAP2K2; STAT3; MAP2K1; BRAF; ATF4 |
| Mitochondrial | SOD2; MAPK8; CASP8; MAPK10; MAPK9; CASP9; |
| Dysfunction | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta | GNAS; GNAQ; PPP2R1A; GNB2L1; PPP2CA; PPP1CC; |
| Adrenergic Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRK1B |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid | PRDX6; GRN; YWHAZ; CYP1B1 |
| Metabolism | |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics | |
| by Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative | |
| Stress Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin Metabolism | TYR |
| Tyrosine Metabolism | PRMT5, TYR |
| Ubiquinone | PRMT5 |
| Biosynthesis | |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; |
| Neurology | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

Therapeutic Use(s)

Systems and compositions of the present disclosure are useful for a variety of applications. For example, systems and methods of the present disclosure are useful in methods of regulating gene expression and/or cellular activity. In an aspect, the systems and compositions disclosed herein are utilized in methods of regulating gene expression and/or cellular activity in an immune cell. Immune cells regulated using a subject system can be useful in a variety of applications, including, but not limited to, immunotherapy to treat diseases and disorders. Diseases and disorders that can be treated using modified immune cells of the present disclosure include inflammatory conditions, cancer, and infectious diseases. In some embodiments, immunotherapy is used to treat cancer.

A variety of target cells can be killed using the systems and methods of the subject disclosure. A target cell to which this method can be applied includes a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a totipotent stem cell, however, in some embodiments of this disclosure, the term “cell” may be used but may not refer to a totipotent stem cell. A target cell can be a plant cell, but in some embodiments of this disclosure, the term “cell” may be used but may not refer to a plant cell. A target cell can be a pluripotent cell. For example, a target cell can be a pluripotent hematopoietic cell that can differentiate into other cells in the hematopoietic cell lineage but may not be able to differentiate into any other non-hematopoietic cell. A target cell may be able to develop into a whole organism. A target cell may or may not be able to develop into a whole organism. A target cell may be a whole organism.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Of particular interest are cancer cells. In some embodiments, the target cell is a cancer cell. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

In some embodiments, the target cells form a tumor. A tumor treated with the methods herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

Death of target cells can be determined by any suitable method, including, but not limited to, counting cells before and after treatment, or measuring the level of a marker associated with live or dead cells (e.g. live or dead target cells). Degree of cell death can be determined by any suitable method. In some embodiments, degree of cell death is determined with respect to a starting condition. For example, an individual can have a known starting amount of target cells, such as a starting cell mass of known size or circulating target cells at a known concentration. In such cases, degree of cell death can be expressed as a ratio of surviving cells after treatment to the starting cell population. In some embodiments, degree of cell death can be determined by a suitable cell death assay. A variety of cell death assays are available, and can utilize a variety of detection methodologies. Examples of detection methodologies include, without limitation, the use of cell staining, microscopy, flow cytometry, cell sorting, and combinations of these.

When a tumor is subject to surgical resection following completion of a therapeutic period, the efficacy of treatment in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%). In some embodiments, the necrosis percentage of the resected tissue is 100%, that is, no living tumor tissue is present or detectable.

In some cases, exposing a target cell to an immune cell or population of immune cells disclosed herein can be conducted either in vitro or in vivo. Exposing a target cell to an immune cell or population of immune cells generally refers to bringing the target cell in contact with the immune cell and/or in sufficient proximity such that an antigen of a target cell (e.g., membrane bound or non-membrane bound) can bind to the ligand interacting domain of the chimeric transmembrane receptor polypeptide expressed in the immune cell. Exposing a target cell to an immune cell or population of immune cells in vitro can be accomplished by co-culturing the target cells and the immune cells. Target cells and immune cells can be co-cultured, for example, as adherent cells or alternatively in suspension. Target cells and immune cells can be co-cultured in various suitable types of cell culture media, for example with supplements, growth factors, ions, etc. Exposing a target cell to an immune cell or population of immune cells in vivo can be accomplished, in some cases, by administering the immune cells to a subject, for example a human subject, and allowing the immune cells to localize to the target cell via the circulatory system. In some cases, an immune cell can be delivered to the immediate area where a target cell is localized, for example, by direct injection.

Exposing can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

In some embodiments, cells expressing a system provided herein induce death of a target cell in an in vitro cell death assay. The cells expressing a system provided herein may exhibit enhanced ability to induce death of the target cell compared to control cells not expressing a system of the present disclosure. In some cases, the enhanced ability to induce death of the target cell is at least a 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, or 1000-fold increase in induced cell death. The degree of induced cell death can be determined at any suitable time point, for example, at least 4 hours, 6 hours, 8 hours, 12 hours, 16 hours, 24 hours, 36 hours, 48 hours, or 52 hours after contacting the cell to the target cell.

In some embodiments, a target polynucleotide can comprise one or more disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissue compared with tissue(s) or cells of a non-disease control. In some embodiments, it is a gene that becomes expressed at an abnormally high level. In some embodiments, it is a gene that becomes expressed at an abnormally low level. The altered expression can correlate with the occurrence and/or progression of the disease. A disease-associated gene also refers to a gene possessing mutation(s) or genetic variation that is directly responsible or is in linkage disequilibrium with a gene(s) that is response for the etiology of a disease. The transcribed or translated products may be known or unknown, and may be at a normal or abnormal level.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web. Exemplary genes associated with certain diseases and disorders are provided in Tables 2 and 3.

Mutations in these genes and pathways can result in production of improper proteins or proteins in improper amounts which affect function.

Promoters that can be used with the methods and compositions of the disclosure include, for example, promoters active in a eukaryotic, mammalian, non-human mammalian or human cell. The promoter can be an inducible or constitutively active promoter. Alternatively or additionally, the promoter can be tissue or cell specific. The promoter can be native or composite promoter.

Non-limiting examples of suitable eukaryotic promoters (i.e. promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF 1), ubiquitin B promoter (UB), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be cell, tissue or tumor specific, such as CD45 promoter, AFP promoter, human Albumin promoter (Alb), MUC1 promoter, COX2 promoter, SP-B promoter, OG-2 promoter. The promoter can be a fungi promoter. The promoter can be a plant promoter. A database of plant promoters can be found (e.g., PlantProm). The expression vector may also contain a ribosome binding site for translation initiation and a transcription terminator. The expression vector may also include appropriate sequences for amplifying expression. Another example of a promoter for the expression vector may include myeloproliferative sarcoma virus enhancer, negative control region deleted, d1587rev primer-binding site substituted (MND) promoter. A promoter for driving RNA can include RNA Pol III promoters (e.g., U6 or H1), Pol II promoters, and/or tRNA(val) promoter.

TABLE 2

| DISEASE/DISORDERS | GENE(S) |
|---|---|
| Neoplasia | PTEN; ATM; ATR; EGFR; ERBB2; ERBB3; ERBB4; |
| | Notch1; Notch2; Notch3; Notch4; AKT; AKT2; AKT3; HIF; |
| | HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; DAOA; |
| | DTNBP1; Dao (Dao1) |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and Atn1 |
| | (DRPLA Dx); CBP (Creb-BP - global instability); VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion - related | Prp |
| disorders | |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's | E1; CHIP; UCH; UBB; Tau; LRP; PICALM; Clusterin; PS1; |
| Disease | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE 3

| | |
|---|---|
| Blood and | Anemia (CDAN1, CDA1, RPS19, DBA, PKLR, PK1, NT5C3, UMPH1, |
| coagulation | PSN1, RHAG, RH50A, NRAMP2, SPTB, ALAS2, ANH1, ASB, |
| diseases and | ABCB7, ABC7, ASAT); Bare lymphocyte syndrome (TAPBP, TPSN, |
| disorders | TAP2, ABCB3, PSF2, RING11, MHC2TA, C2TA, RFX5, RFXAP, |
| | RFX5), Bleeding disorders (TBXA2R, P2RX1, P2X1); Factor H and |
| | factor H-like 1 (HF1, CFH, HUS); Factor V and factor VIII (MCFD2); |
| | Factor VII deficiency (F7); Factor X deficiency (F10); Factor XI |
| | deficiency (F11); Factor XII deficiency (F12, HAF); Factor XIIIA |
| | deficiency (F13A1, F13A); Factor XIIIB deficiency (F13B); Fanconi |
| | anemia (FANCA, FACA, FA1, FA, FAA, FAAP95, FAAP90, FLJ34064, |
| | FANCB, FANCC, FACC, BRCA2, FANCD1, FANCD2, FANCD, |
| | FACD, FAD, FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, HPLH2, |
| | UNC13D, MUNC13-4, HPLH3, HLH3, FHL3); Hemophilia A (F8, F8C, |
| | HEMA); Hemophilia B (F9, HEMB), Hemorrhagic disorders (PI, ATT, |
| | F5); Leukocyde deficiencies and disorders (ITGB2, CD18, LCAMB, |
| | LAD, EIF2B1, EIF2BA, EIF2B2, EIF2B3, EIF2B5, LVWM, CACH, |
| | CLE, EIF2B4); Sickle cell anemia (HBB); Thalassemia (HBA2, HBB, |
| | HBD, LCRB, HBA1). |
| Cell | B-cell non-Hodgkin lymphoma (BCL7A, BCL7); Leukemia (TAL1, |
| dysregulation | TCL5, SCL, TAL2, FLT3, NBS1, NBS, ZNFN1A1, IK1, LYF1, |
| and oncology | HOXD4, HOX4B, BCR, CML, PHL, ALL, ARNT, KRAS2, RASK2, |
| diseases and | GMPS, AF10, ARHGEF12, LARG, KIAA0382, CALM, CLTH, |
| disorders | CEBPA, CEBP, CHIC2, BTL, FLT3, KIT, PBT, LPP, NPM1, NUP214, |
| | D9S46E, CAN, CAIN, RUNX1, CBFA2, AML1, WHSC1L1, NSD3, |
| | FLT3, AF1Q, NPM1, NUMA1, ZNF145, PLZF, PML, MYL, STAT5B, |
| | AF10, CALM, CLTH, ARL11, ARLTS1, P2RX7, P2X7, BCR, CML, |
| | PHL, ALL, GRAF, NF1, VRNF, WSS, NFNS, PTPN11, PTP2C, SHP2, |
| | NS1, BCL2, CCND1, PRAD1, BCL1, TCRA, GATA1, GF1, ERYF1, |
| | NFE1, ABL1, NQO1, DIA4, NMOR1, NUP214, D9S46E, CAN, CAIN). |
| Inflammation | AIDS (KIR3DL1, NKAT3, NKB1, AMB11, KIR3DS1, IFNG, CXCL12, |
| and immune | SDF1); Autoimmune lymphoproliferative syndrome (TNFRSF6, APT1, |
| related | FAS, CD95, ALPS1A); Combined immunodeficiency, (IL2RG, |
| diseases and | SCIDX1, SCIDX, IMD4); HIV-1 (CCL5, SCYA5, D17S136E, TCP228), |
| disorders | HIV susceptibility or infection (IL10, CSIF, CMKBR2, CCR2, |
| | CMKBR5, CCCKR5 (CCR5)); Immunodeficiencies (CD3E, CD3G, |
| | AICDA, AID, HIGM2, TNFRSF5, CD40, UNG, DGU, HIGM4, |
| | TNFSF5, CD40LG, HIGM1, IGM, FOXP3, IPEX, AIID, XPID, PIDX, |
| | TNFRSF14B, TACI); Inflammation (IL-10, IL-1 (IL-1a, IL-1b), IL-13, |
| | IL-17 (IL-17a (CTLA8), IL-17b, IL-17c, IL-17d, IL-17f), II-23, Cx3cr1, |
| | ptpn22, TNFa, NOD2/CARD 15 for IBD, IL-6, IL-12 (IL-12a, IL-12b), |
| | CTLA4, Cx3cl1); Severe combined immunodeficiencies (SCIDs)(JAK3, |
| | JAKL, DCLRE1C, ARTEMIS, SCIDA, RAG1, RAG2, ADA, PTPRC, |
| | CD45, LCA, IL7R, CD3D, T3D, IL2RG, SCIDX1, SCIDX, IMD4). |
| Metabolic, | Amyloid neuropathy (TTR, PALB); Amyloidosis (APOA1, APP, AAA, |
| liver, | CVAP, AD1, GSN, FGA, LYZ, TTR, PALB); Cirrhosis (KRT18, KRT8, |
| kidney and | CIRH1A, NAIC, TEX292, KIAA1988); Cystic fibrosis (CFTR, ABCC7, |
| protein | CF, MRP7); Glycogen storage diseases (SLC2A2, GLUT2, G6PC, |
| diseases and | G6PT, G6PT1, GAA, LAMP2, LAMPB, AGL, GDE, GBE1, GYS2, |
| disorders | PYGL, PFKM); Hepatic adenoma, 142330 (TCF1, HNF1A, MODY3), |
| | Hepatic failure, early onset, and neurologic disorder (SCOD1, SCO1), |
| | Hepatic lipase deficiency (LIPC), Hepatoblastoma, cancer and |
| | carcinomas (CTNNB1, PDGFRL, PDGRL, PRLTS, AXIN1, AXIN, |
| | CTNNB1, TP53, P53, LFS1, IGF2R, MPRI, MET, CASP8, MCH5; |
| | Medullary cystic kidney disease (UMOD, HNFJ, FJHN, MCKD2, |
| | ADMCKD2); Phenylketonuria (PAH, PKU1, QDPR, DHPR, PTS); |
| | Polycystic kidney and hepatic disease (FCYT, PKHD1, ARPKD, PKD1, |
| | PKD2, PKD4, PKDTS, PRKCSH, G19P1, PCLD, SEC63). |
| Muscular/ | Becker muscular dystrophy (DMD, BMD, MYF6), Duchenne Muscular |
| Skeletal | Dystrophy (DMD, BMD); Emery-Dreifuss muscular dystrophy (LMNA, |
| diseases and | LMN1, EMD2, FPLD, CMD1A, HGPS, LGMD1B, LMNA, LMN1, |
| disorders | EMD2, FPLD, CMD1A); Facioscapulohumeral muscular dystrophy |
| | (FSHMD1A, FSHD1A); Muscular dystrophy (FKRP, MDC1C, |
| | LGMD2I, LAMA2, LAMM, LARGE, KIAA0609, MDC1D, FCMD, |
| | TTID, MYOT, CAPN3, CANP3, DYSF, LGMD2B, SGCG, LGMD2C, |
| | DMDA1, SCG3, SGCA, ADL, DAG2, LGMD2D, DMDA2, SGCB, |
| | LGMD2E, SGCD, SGD, LGMD2F, CMD1L, TCAP, LGMD2G, |
| | CMD1N, TRIM32, HT2A, LGMD2H, FKRP, MDC1C, LGMD2I, TTN, |
| | CMD1G, TMD, LGMD2J, POMT1, CAV3, LGMD1C, SEPN1, SELN, |
| | RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, |
| | TIRC7, OC116, OPTB1); Muscular atrophy (VAPB, VAPC, ALS8, |
| | SMN1, SMA1, SMA2, SMA3, SMA4, BSCL2, SPG17, GARS, SMAD1, |
| | CMT2D, HEXB, IGHMBP2, SMUBP2, CATF1, SMARD1). |
| Neurological | ALS (SOD1, ALS2, STEX, FUS, TARDBP, VEGF (VEGF-a, VEGF-b, |
| and neuronal | VEGF-c); Alzheimer disease (APP, AAA, CVAP, AD1, APOE, AD2, |
| diseases and | PSEN2, AD4, STM2, APBB2, FE65L1, NOS3, PLAU, URK, ACE, |
| disorders | DCP1, ACE1, MPO, PACIP1, PAXIP1L, PTIP, A2M, BLMH, BMH, |
| | PSEN1, AD3); Autism (Mecp2, BZRAP1, MDGA2, Sema5A, Neurexin |
| | 1, GLO1, MECP2, RTT, PPMX, MRX16, MRX79, NLGN3, NLGN4, |
| | KIAA1260, AUTSX2); Fragile X Syndrome (FMR2, FXR1, FXR2, |
| | mGLUR5); Huntington's disease and disease like disorders (HD, IT15, |
| | PRNP, PRIP, JPH3, JP3, HDL2, TBP, SCA17); Parkinson disease |
| | (NR4A2, NURR1, NOT, TINUR, SNCAIP, TBP, SCA17, SNCA, |
| | NACP, PARK1, PARK4, DJ1, PARK7, LRRK2, PARK8, PINK1, |
| | PARK6, UCHL1, PARK5, SNCA, NACP, PARK1, PARK4, PRKN, |
| | PARK2, PDJ, DBH, NDUFV2); Rett syndrome (MECP2, RTT, PPMX, |
| | MRX16, MRX79, CDKL5, STK9, MECP2, RTT, PPMX, MRX16, |
| | MRX79, x-Synuclein, DJ-1); Schizophrenia (Neuregulin1 (Nrg1), Erb4 |
| | (receptor for Neuregulin), Complexin1 (Cplx1), Tph1 Tryptophan |
| | hydroxylase, Tph2, Tryptophan hydroxylase 2, Neurexin 1, GSK3, |
| | GSK3a, GSK3b, 5-HTT (Slc6a4), COMT, DRD (Drd1a), SLC6A3, |
| | DAOA, DTNBP1, Dao (Dao1)); Secretase Related Disorders (APH-1 |
| | (alpha and beta), Presenilin (Psen1), nicastrin, (Ncstn), PEN-2, Nos1, |
| | Parp1, Nat1, Nat2); Trinucleotide Repeat Disorders (HTT (Huntington's |
| | Dx), SBMA/SMAX1/AR (Kennedy's Dx), FXN/X25 (Friedrich's |
| | Ataxia), ATX3 (Machado- Joseph's Dx), ATXN1 and ATXN2 |
| | (spinocerebellar ataxias), DMPK (myotonic dystrophy), Atrophin-1 and |
| | Atn1 (DRPLA Dx), CBP (Creb-BP - global instability), VLDLR |
| | (Alzheimer's), Atxn7, Atxn10). |
| Occular | Age-related macular degeneration (Abcr, Ccl2, Cc2, cp (ceruloplasmin), |
| diseases and | Timp3, cathepsinD, Vldlr, Ccr2); Cataract (CRYAA, CRYA1, CRYBB2, |
| disorders | CRYB2, PITX3, BFSP2, CP49, CP47, CRYAA, CRYA1, PAX6, AN2, |
| | MGDA, CRYBA1, CRYB1, CRYGC, CRYG3, CCL, LIM2, MP19, |
| | CRYGD, CRYG4, BFSP2, CP49, CP47, HSF4, CTM, HSF4, CTM, |
| | MIP, AQP0, CRYAB, CRYA2, CTPP2, CRYBB1, CRYGD, CRYG4, |
| | CRYBB2, CRYB2, CRYGC, CRYG3, CCL, CRYAA, CRYA1, GJA8, |
| | CX50, CAE1, GJA3, CX46, CZP3, CAE3, CCM1, CAM, KRIT1); |
| | Corneal clouding and dystrophy (APOA1, TGFBI, CSD2, CDGG1, |
| | CSD, BIGH3, CDG2, TACSTD2, TROP2, M1S1, VSX1, RINX, PPCD, |
| | PPD, KTCN, COL8A2, FECD, PPCD2, PIP5K3, CFD); Cornea plana |
| | congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, |
| | NTG, NPG, CYP1B1, GLC3A); Leber congenital amaurosis (CRB1, |
| | RP12, CRX, CORD2, CRD, RPGRIP1, LCA6, CORD9, RPE65, RP20, |
| | AIPL1, LCA4, GUCY2D, GUC2D, LCA1, CORD6, RDH12, LCA3); |
| | Macular dystrophy (ELOVL4, ADMD, STGD2, STGD3, RDS, RP7, |
| | PRPH2, PRPH, AVMD, AOFMD, VMD2). |

Systems and compositions of the present disclosure are useful for other varieties of applications. For example, systems and methods of the present disclosure are useful in methods of regulating gene expression and/or cellular activity critical for cell proliferation, differentiation, trans-differentiation, and/or de-differentiation during tissue (e.g., an organ) growth, repair, regeneration, regenerative medicine, and/or engineering. Examples of the tissue include epithelial, connective, nerve, muscle, organ, and other tissues. Other exemplary tissues include artery, ligament, skin, tendon, kidney, nerve, liver, pancreas, bladder, bone, lung, blood vessels, heart valve, cartilage, eyes, etc.

### EXAMPLES

Various aspects of the disclosure are further illustrated by the following non-limiting examples.

### Example 1: Formation of a Signaling Complex of a Receptor by a First Chimeric Polypeptide and a Second Chimeric Polypeptide

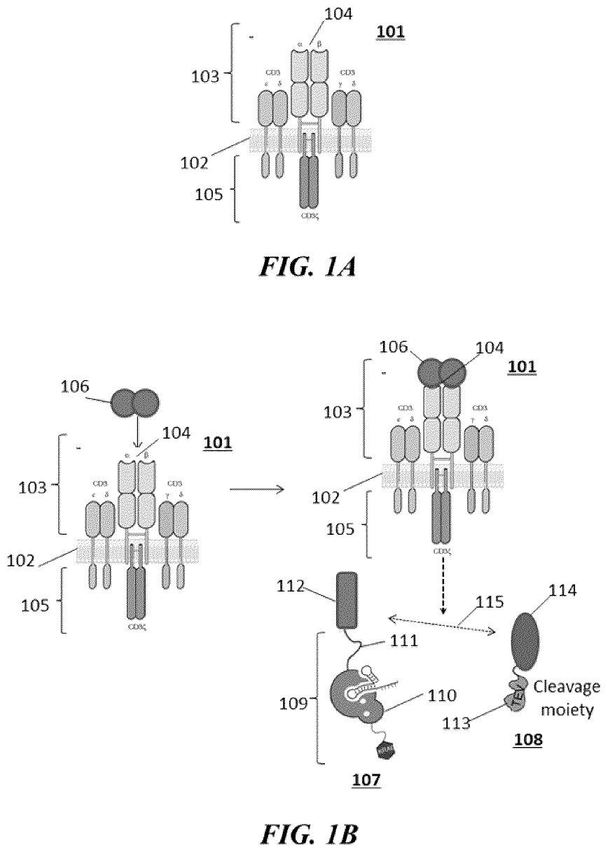

FIGS. 1A-1B illustrate schematically the formation of a signaling complex of a receptor (or receptor complex). FIG. 1A shows a transmembrane receptor polypeptide 101 spanning membrane 102. The transmembrane receptor polypeptide comprises an extracellular region 103 having a ligand binding domain 104 and an intracellular region 105. FIG. 1B shows the binding of a ligand 106 to the ligand binding domain 104 to induce the formation of a signaling complex of the receptor. The signaling complex comprises a first chimeric polypeptide 107 and a second chimeric polypeptide 108. The first chimeric polypeptide 107 comprises a gene modulating polypeptide (GMP) 109 comprising actuator moiety 110 capable of regulating expression and/or activity of a target gene or edit a nucleic acid sequence in the cell. The actuator moiety 110 is linked to a cleavage recognition site 111. The cleavage recognition site 111 is flanked by a first adaptor moiety 112 and the actuator moiety 110. The second chimeric polypeptide 108 comprises a cleavage moiety 113 linked to a second adaptor moiety 114. The cleavage moiety 113 may be complexed with the second adaptor moiety 114 or linked, for example, by a peptide bond and/or peptide linker, to the second adaptor moiety 114. The cleavage moiety 113 is capable of cleaving the cleavage recognition site 111 of the GMP 109. In response to the binding of the ligand 106 to the ligand binding domain 104, the transmembrane receptor polypeptide 101 is modified in the intracellular region 105. Following receptor modification (e.g., phosphorylation), the first chimeric polypeptide 107 and the second chimeric polypeptide 108 may bind to each other 115 or come in proximity to each other 115 sufficient for inducing the action of the cleavage moiety 113 to cleave and release the actuator moiety 110 from the first adaptor moiety 112. Upon release, the actuator moiety 110 can translocate (e.g., enter a nucleus) to regulate the expression and/or activity of a target gene or edit a nucleic acid sequence.

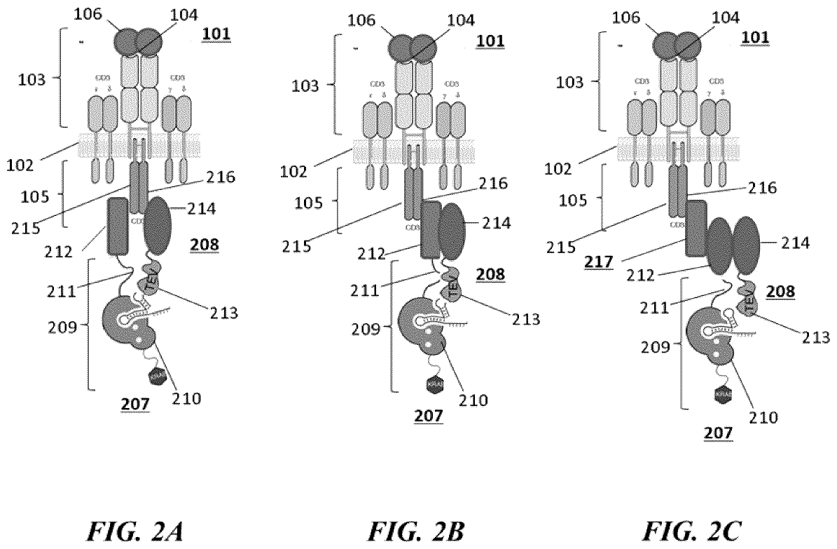

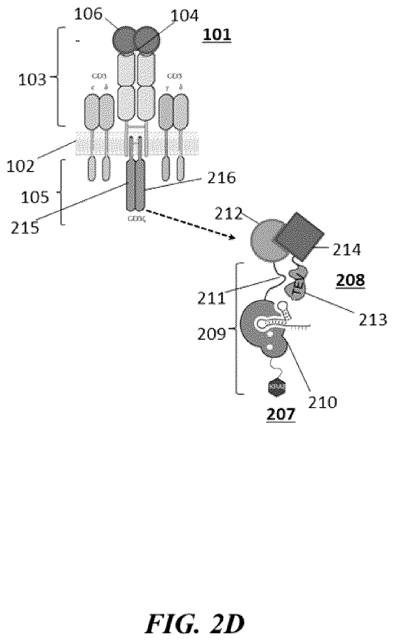

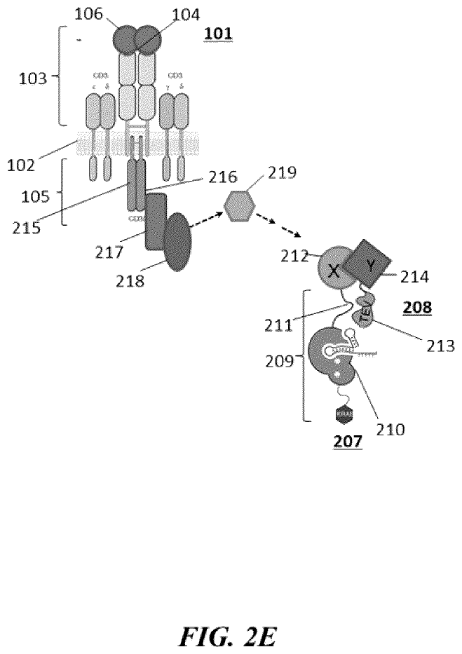

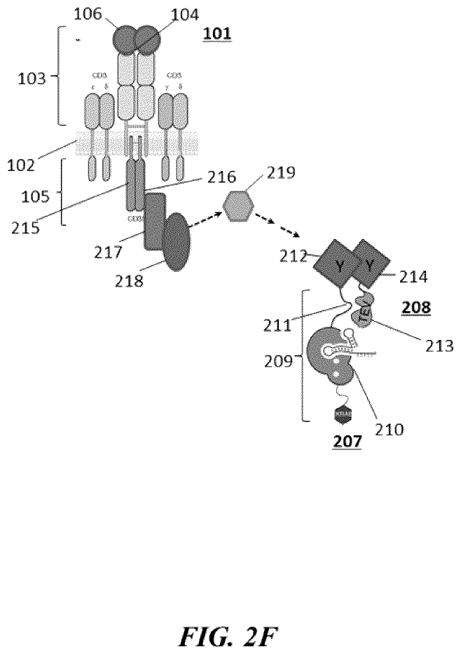

FIGS. 2A-2F illustrate schematically various configurations of a signaling complex of a transmembrane receptor polypeptide. FIG. 2A shows a signaling complex in which a first chimeric polypeptide 207 and a second chimeric polypeptide 208 interact with an intracellular region 105 of a transmembrane receptor polypeptide 101 spanning membrane 102. The transmembrane receptor polypeptide 101 comprises an extracellular region 103 having a ligand binding domain 104 and the intracellular region 105. The transmembrane receptor polypeptide 101 comprises a first intracellular domain 215 and a second intracellular domain 216. Upon binding of a ligand 106 to the ligand binding domain 104, the transmembrane receptor polypeptide 101 is modified in the intracellular region 105. Following receptor modification (e.g., phosphorylation), the first chimeric polypeptide 207 and the second chimeric polypeptide 208 come in proximity to each other, thereby forming a signaling complex.

Referring to FIG. 2A, the first chimeric polypeptide 207 comprises a gene modulating polypeptide (GMP) 209 comprising actuator moiety 210 capable of regulating expression and/or activity of a target gene or edit a nucleic acid sequence in the cell. The actuator moiety 210 is linked to a cleavage recognition site 211. The cleavage recognition site 211 is flanked by a first adaptor moiety 212 and the actuator moiety 210. The first adaptor moiety 212 directly binds to the first intracellular domain 215. The second chimeric polypeptide 208 comprises a cleavage moiety 213 linked to a second adaptor moiety 214. The second adaptor moiety 214 directly binds to the second intracellular domain 216. The cleavage moiety 213 may be complexed with the second adaptor moiety 214 or linked, for example, by a peptide bond and/or peptide linker, to the second adaptor moiety 214. The cleavage moiety 213 is capable of cleaving the cleavage recognition site 211 of the GMP 209, thereby releasing the actuator moiety 210, which enters the nucleus to regulate the expression and/or activity of the target gene or edit the nucleic acid sequence.

FIG. 2B shows a signaling complex in which a first chimeric polypeptide 207 and a second chimeric polypeptide 208 interact with an intracellular region 105 of a transmembrane receptor polypeptide 101 spanning membrane 102. The transmembrane receptor polypeptide 101 comprises an extracellular region 103 having a ligand binding domain 104 and the intracellular region 105. The transmembrane receptor polypeptide 101 comprises a first intracellular domain 215 and a second intracellular domain 216. Upon binding of a ligand 106 to the ligand binding domain 104, the transmembrane receptor polypeptide 101 is modified in the intracellular region 105. Following receptor modification (e.g., phosphorylation), the first chimeric polypeptide 207 and the second chimeric polypeptide 208 bind to each other, thereby forming a signaling complex.

Referring to FIG. 2B, the first chimeric polypeptide 207 comprises a gene modulating polypeptide (GMP) 209 comprising actuator moiety 210 capable of regulating expression and/or activity of a target gene or edit a nucleic acid sequence in the cell. The actuator moiety 210 is linked to a cleavage recognition site 211. The cleavage recognition site 211 is flanked by a first adaptor moiety 212 and the actuator moiety 210. The first adaptor moiety 212 directly binds to the second intracellular domain 216. The second chimeric polypeptide 208 comprises a cleavage moiety 213 linked to a second adaptor moiety 214. The second adaptor moiety 214 directly binds to the first adaptor moiety 212. The second adaptor moiety 214 does not directly bind to the second intracellular domain 216 or the intracellular region 105. The cleavage moiety 213 may be complexed with the second adaptor moiety 214 or linked, for example by a peptide bond and/or peptide linker, to the second adaptor moiety 214. The cleavage moiety 213 is capable of cleaving the cleavage recognition site 211 of the GMP 209, thereby releasing the actuator moiety 210, which enters the nucleus to regulate the expression and/or activity of the target gene or edit the nucleic acid sequence.

FIG. 2C shows a signaling complex in which a first chimeric polypeptide 207, a second chimeric polypeptide 208, and a first signaling moiety 217 interact with an intracellular region 105 of a transmembrane receptor polypeptide 101 spanning membrane 102. The transmembrane receptor polypeptide 101 comprises an extracellular region 103 having a ligand binding domain 104 and the intracellular region 105. The transmembrane receptor polypeptide 101 comprises a first intracellular domain 215 and a second intracellular domain 216. Upon binding of a ligand 106 to the ligand binding domain 104, the transmembrane receptor polypeptide 101 is modified in the intracellular region 105. Following receptor modification (e.g., phosphorylation), the first signaling moiety 217 binds to the second intracellular domain 216 of the intracellular region 105, which first signaling moiety 217 also binds to the first chimeric polypeptide 207 that binds to the second chimeric polypeptide 208, thereby forming a signaling complex.

Referring to FIG. 2C, the first chimeric polypeptide 207 comprises a gene modulating polypeptide (GMP) 209 comprising actuator moiety 210 capable of regulating expression and/or activity of a target gene or edit a nucleic acid sequence in the cell. The actuator moiety 210 is linked to a cleavage recognition site 211. The cleavage recognition site 211 is flanked by a first adaptor moiety 212 and the actuator moiety 210. The first adaptor moiety 212 directly binds to the first signaling moiety 217, which directly binds to the second intracellular domain 216. The second chimeric polypeptide 208 comprises a cleavage moiety 213 linked to a second adaptor moiety 214. The second adaptor moiety 214 directly binds to the first adaptor moiety 212. The cleavage moiety 213 may be complexed with the second adaptor moiety 214 or linked, for example, by a peptide bond and/or peptide linker, to the second adaptor moiety 214. The cleavage moiety 213 is capable of cleaving the cleavage recognition site 211 of the GMP 209, thereby releasing the actuator moiety 210, which enters the nucleus to regulate the expression and/or activity of the target gene or edit the nucleic acid sequence.

FIG. 2D shows a signaling complex in which a first chimeric polypeptide 207 and a second chimeric polypeptide 208 interact with each other during signaling of a transmembrane receptor polypeptide 101 spanning membrane 102. The transmembrane receptor polypeptide 101 comprises an extracellular region 103 having a ligand binding domain 104 and the intracellular region 105. The transmembrane receptor polypeptide 101 comprises a first intracellular domain 215 and a second intracellular domain 216. Upon binding of a ligand 106 to the ligand binding domain 104, the transmembrane receptor polypeptide 101 is modified in the intracellular region 105. Following receptor modification (e.g., phosphorylation), one or more intracellular signaling cascade is activated, during which the first chimeric polypeptide 207 and the second chimeric polypeptide 208 interact with each other, thereby forming a signaling complex.

Referring to FIG. 2D, the first chimeric polypeptide 207 comprises a gene modulating polypeptide (GMP) 209 comprising actuator moiety 210 capable of regulating expression and/or activity of a target gene or edit a nucleic acid sequence in the cell. The actuator moiety 210 is linked to a cleavage recognition site 211. The cleavage recognition site 211 is flanked by a first adaptor moiety 212 and the actuator moiety 210. The first adaptor moiety 212 is activatable to be or to bind a first downstream signaling moiety of the receptor. The second chimeric polypeptide 208 comprises a cleavage moiety 213 linked to a second adaptor moiety 214. The cleavage moiety 213 may be complexed with the second adaptor moiety 214 or linked, for example by a peptide bond and/or peptide linker, to the second adaptor moiety 214. The second adaptor moiety 214 is activatable to bind the first adaptor moiety 212. Upon binding of a ligand 106 to the ligand binding domain 104, the first chimeric polypeptide 207 and the second chimeric polypeptide 208 are not in direct contact with the receptor but are in association with each other at downstream of the signaling of the transmembrane receptor polypeptide 101. The cleavage moiety 213 is capable of cleaving the cleavage recognition site 211 of the GMP 209, thereby releasing the actuator moiety 210, which enters the nucleus to regulate the expression and/or activity of the target gene or edit the nucleic acid sequence.

FIG. 2E shows a signaling complex in which a first chimeric polypeptide 207 and a second chimeric polypeptide 208 interact with each other during signaling of a transmembrane receptor polypeptide 101 spanning membrane 102. The transmembrane receptor polypeptide 101 comprises an extracellular region 103 having a ligand binding domain 104 and the intracellular region 105. The transmembrane receptor polypeptide 101 comprises a first intracellular domain 215 and a second intracellular domain 216. Upon binding of a ligand 106 to the ligand binding domain 104, the transmembrane receptor polypeptide 101 is modified in the intracellular region 105. Following receptor modification (e.g., phosphorylation), the first chimeric polypeptide 207 and the second chimeric polypeptide 208 bind to each other, thereby forming a signaling complex.

Referring to FIG. 2E, The first chimeric polypeptide 207 comprises a gene modulating polypeptide (GMP) 209 comprising actuator moiety 210 capable of regulating expression and/or activity of a target gene or edit a nucleic acid sequence in the cell. The actuator moiety 210 is linked to a cleavage recognition site 211. The cleavage recognition site 211 is flanked by a first adaptor moiety 212 and the actuator moiety 210. The second chimeric polypeptide 208 comprises a cleavage moiety 213 linked to a second adaptor moiety 214. The cleavage moiety 213 may be complexed with the second adaptor moiety 214 or linked, for example by a peptide bond and/or peptide linker, to the second adaptor moiety 214. The second adaptor moiety 214 is activatable to bind the first adaptor moiety 212. The first adaptor moiety 212 and the second adaptor moiety 214 are different moieties. Upon binding of a ligand 106 to the ligand binding domain 104, a cellular response may be initiated using at least one signaling cascade involving additional proteins such as a first signaling moiety 217 that binds directly to the second intracellular domain 216, and a second signaling moiety 218 that binds directly to the first signaling moiety 217. The signaling cascade may involve a third signaling moiety 219, which recruits the first chimeric polypeptide 207 and the second chimeric polypeptide 208 to be in association with each other downstream of the signaling of the transmembrane receptor polypeptide 101. The cleavage moiety 213 is capable of cleaving the cleavage recognition site 211 of the GMP 209, thereby releasing the actuator moiety 210, which enters the nucleus to regulate the expression and/or activity of the target gene or edit the nucleic acid sequence.

FIG. 2F shows a signaling complex in which a first chimeric polypeptide 207 and a second chimeric polypeptide 208 interact with each other during signaling of a transmembrane receptor polypeptide 101 spanning membrane 102. The transmembrane receptor polypeptide 101 comprises an extracellular region 103 having a ligand binding domain 104 and the intracellular region 105. The transmembrane receptor polypeptide 101 comprises a first intracellular domain 215 and a second intracellular domain 216. Upon binding of a ligand 106 to the ligand binding domain 104, the transmembrane receptor polypeptide 101 is modified in the intracellular region 105. Following receptor modification (e.g., phosphorylation), the first chimeric polypeptide 207 and the second chimeric polypeptide 208 bind to each other, thereby forming a signaling complex.

Referring to FIG. 2F, the first chimeric polypeptide 207 comprises a gene modulating polypeptide (GMP) 209 comprising actuator moiety 210 capable of regulating expression and/or activity of a target gene or edit a nucleic acid sequence in the cell. The actuator moiety 210 is linked to a cleavage recognition site 211. The cleavage recognition site 211 is flanked by a first adaptor moiety 212 and the actuator moiety 210. The second chimeric polypeptide 208 comprises a cleavage moiety 213 linked to a second adaptor moiety 214. The cleavage moiety 213 may be complexed with the second adaptor moiety 214 or linked, for example by a peptide bond and/or peptide linker, to the second adaptor moiety 214. The second adaptor moiety 214 is activatable to bind the first adaptor moiety 212. The first adaptor moiety 212 and the second adaptor moiety 214 are the same moieties. Upon binding of a ligand 106 to the ligand binding domain 104, a cellular response may be initiated using at least one signaling cascade involving additional proteins such as a first signaling moiety 217 that binds directly to the second intracellular domain 216, and a second signaling moiety 218 that binds directly to the first signaling moiety 217. The signaling cascade may involve a third signaling moiety 219, which recruits the first chimeric polypeptide 207 and the second chimeric polypeptide 208 to be in association with each other downstream of the signaling of the transmembrane receptor polypeptide 101. The cleavage moiety 213 is capable of cleaving the cleavage recognition site 211 of the GMP 209, thereby releasing the actuator moiety 210, which enters the nucleus to regulate the expression and/or activity of the target gene or edit the nucleic acid sequence.

### Example 2: Formation of a Signaling Complex of an Endogenous Receptor by a First Chimeric Polypeptide and a Second Chimeric Polypeptide, Lentivirus

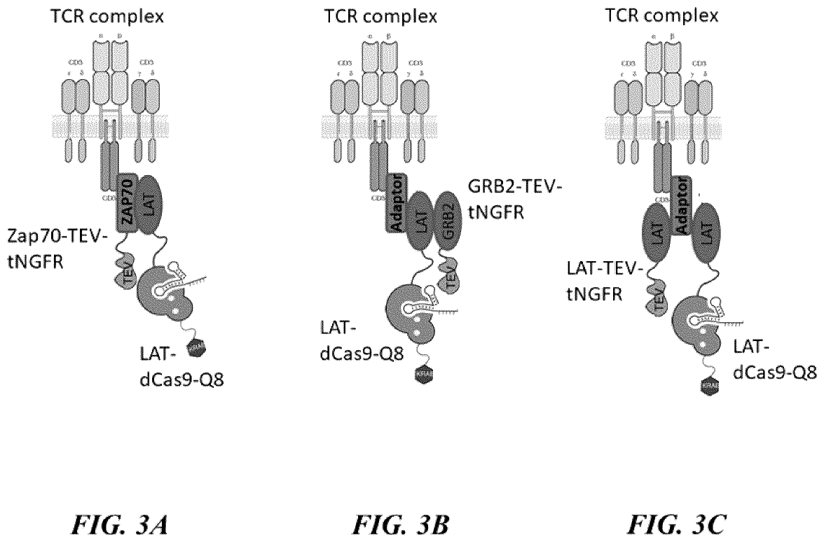

FIGS. 3A-3C schematically illustrates various embodiments of a first chimeric polypeptide and a second chimeric polypeptide that form a complex (e.g., direct complexation, indirect complexation) upon signaling of an endogenous receptor of a cell (e.g., endogenous T cell receptor (TCR) of a T cell). For example, the chimeric polypeptides comprises at least a portion of an adaptor polypeptide that is recruited to or towards the endogenous receptor (e.g., intracellular portion of the endogenous receptor) upon activation of the endogenous receptor (e.g., upon binding of an antigen to the endogenous receptor). The first chimeric polypeptide and the second chimeric polypeptide can comprise the same adaptor polypeptide or different adaptor polypeptides. One of the first chimeric polypeptide and the second chimeric polypeptide comprises a gene modulating polypeptide (GMP) comprising an actuator moiety, while the other of the first chimeric polypeptide and the second chimeric polypeptide comprises a cleavage moiety capable of releasing the actuator moiety from the GMP upon complexation of the first chimeric polypeptide and the second chimeric polypeptide. In some cases, the actuator moiety may not promote regulation of a target gene within the cell (e.g., to a detectable level) in absence of such complexation. In some cases, the first chimeric polypeptide and/or the second chimeric polypeptide can be intracellular polypeptides. Alternatively or in addition to, the first chimeric polypeptide and/or the second chimeric polypeptide can be membrane-bound polypeptides and can be recruited to or towards the endogenous receptor upon activation of the endogenous receptor.

Referring to FIG. 3A, the cell is a T cell and the endogenous receptor is TCR. The first chimeric polypeptide comprises Zeta Chain of T Cell Receptor Associated Protein Kinase 70 (ZAP70) that is coupled to a Tobacco Etch Virus (TEV) protease cleavage moiety, optionally operatively coupled to a reporter (e.g., truncated rat nerve growth factor receptor (tNGFR)). The second chimeric polypeptide comprises Linker for Activation of T Cells (LAT) that is coupled to a Cas enzyme or a modification thereof (e.g., dCas-KRAB), optionally operatively coupled to a reporter (e.g., Q8). Upon activation of the TCR, the first chimeric polypeptide binds to an intracellular portion of the TCR, and the second chimeric polypeptide binds to the first chimeric polypeptide (e.g., via binding between ZAP70 and LAT), to promote TEV to release the actuator moiety from the second chimeric polypeptide.

Referring to FIG. 3B, the cell is a T cell and the endogenous receptor is TCR. The first chimeric polypeptide comprises Growth Factor Receptor Bound Protein 2 (GRB2) that is coupled to a Tobacco Etch Virus (TEV) protease cleavage moiety, optionally operatively coupled to a reporter (e.g., truncated rat nerve growth factor receptor (tNGFR)). The second chimeric polypeptide comprises Linker for Activation of T Cells (LAT) that is coupled to a Cas enzyme or a modification thereof (e.g., dCas-KRAB), optionally operatively coupled to a reporter (e.g., Q8). Upon activation of the TCR, an adaptor protein of the TCR binds to an intracellular portion of the TCR, and the second chimeric polypeptide is recruited towards the TCR via binding between the adaptor protein and the LAT. In addition, the second chimeric polypeptide recruits the first chimeric polypeptide via binding between the LAT and the GRB2, to promote TEV to release the actuator moiety from the second chimeric polypeptide.

Referring to FIG. 3C, the cell is a T cell and the endogenous receptor is TCR. The first chimeric polypeptide comprises Linker for Activation of T Cells (LAT) that is coupled to a Tobacco Etch Virus (TEV) protease cleavage moiety, optionally operatively coupled to a reporter (e.g., truncated rat nerve growth factor receptor (tNGFR)). The second chimeric polypeptide comprises Linker for Activation of T Cells (LAT) that is coupled to a Cas enzyme or a modification thereof (e.g., dCas-KRAB), optionally operatively coupled to a reporter (e.g., Q8). Upon activation of the TCR, an adaptor protein of the TCR binds to an intracellular portion of the TCR, and the adaptor protein recruits (1) the first chimeric polypeptide via binding between the adaptor protein and LAT and (2) the second chimeric polypeptide via binding between the adaptor protein and LAT, to promote TEV to release the actuator moiety from the second chimeric polypeptide.

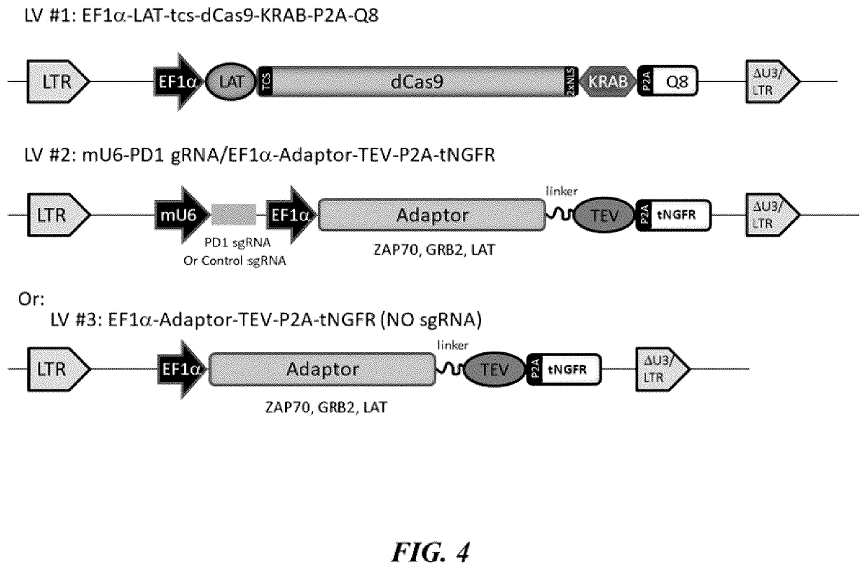

FIG. 4 schematically illustrates expression cassettes (e.g., viral constructs, such as lentiviral constructs) encoding any one of the chimeric polypeptides disclosed herein (e.g., the first chimeric polypeptide or the second chimeric polypeptide). For example, a first lentiviral construct may be a LV #1 construct encoding EF1a-LAT-tcs-dCas9-KRAB-P2A-Q8. In another example, a second lentiviral construct may be a LV #2 construct encoding mU6-PD1 gRNA/EF1a-Adaptor-TEV-P2A-tNGFR. In a different example, a third lentiviral construct may be LV #3 construct encoding EF1a-Adaptor-TEV-P2A-tNGFR, without encoding any sgRNA.

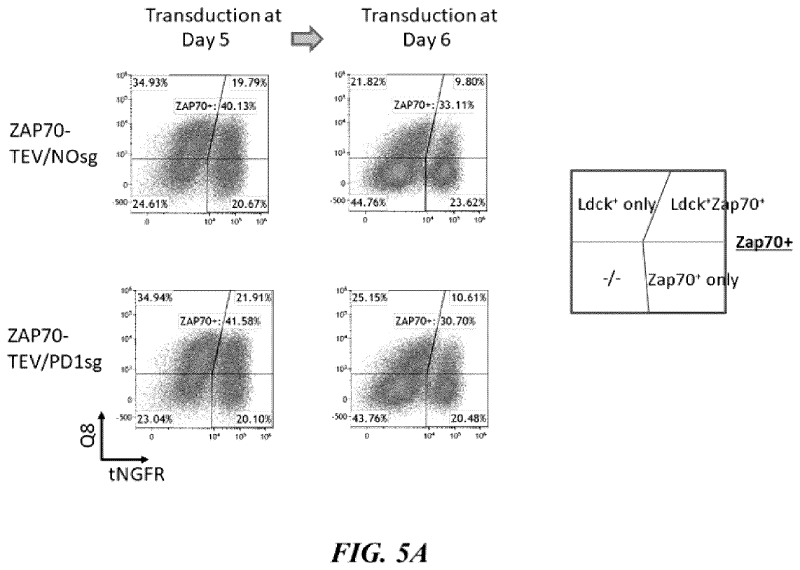

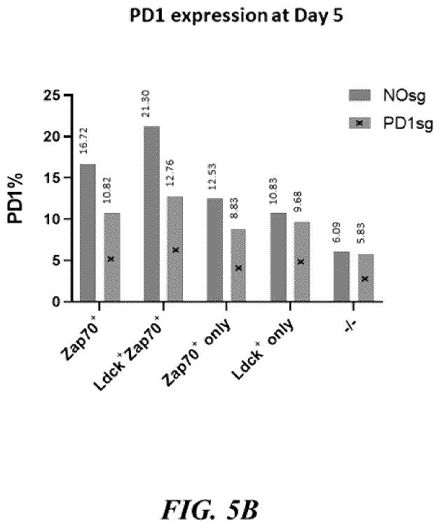

As shown in FIG. 5, human primary T cells were activated by an antigen of TCR (e.g., OKT3/CD28) at day 0. Following, the human primary T cells were transduced with the first lentiviral construct (encoding LAT-tcs-dCas9-KRAB-P2A-Q8 (“Ldck”)) as shown in FIG. 4 at day 1. Following, the human primary T cells were transduced with the second lentiviral construct (encoding PD1 gRNA (“PD1sg”) and Zap70-TEV-P2A-tNGFR (“Zap70”)) or the third lentiviral construct (encoding Zap70-TEV-P2A-tNGFR, but not sgRNA (“NOsg”)) as shown in FIG. 4 at day 2. Referring to FIG. 5A, expression levels of Q8 and tNGFR of the engineered human primary T cells were analyzed by flow cytometry at day 5 and day 6, for both PD1sg and NOsg groups, for in silico sorting of the human primary T cells into the following groups: Zap70+ (a combination of Ldck+Zap70+ and Zap70+ only), Ldck+Zap70+, Zap70+ only, Ldck+ only, or double negative (−/−) human primary T cells. The in silico sorting was based on a predetermined threshold expression level of Q8 and tNGFR in the cells. Referring to FIG. 5B, PD1 expression levels of the engineered human primary T cells were measured (e.g., by flow cytometry) at day 5 and plotted for each in silico sorted group, to compare the differences between NOsg and PD1sg conditions. In this case, the human primary T cells were not activated by a TCR receptor after day 0. Upon the single activation on day 0, the engineered human primary T cells comprising both Ldck construct and Zap70 construct exhibited the greatest decrease (e.g., about 40% decrease) in the expression level of PD1 (or the greatest decrease in the proportion of the cells expressing PD1) between NOsg and PD1sg conditions, as compared to other control cells. Without wishing to be bound by theory, the initial activation of the cells at day 0 may be prolonged to promote continued activation of the TCR, thereby promoting complexation of the chimeric polypeptides to promote activation of the dCas9-KRAB actuator moiety.

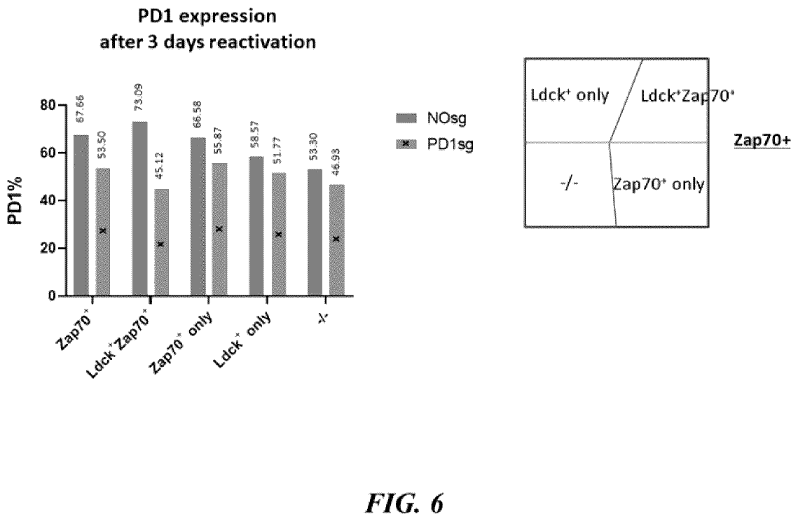

As shown in FIG. 6, the engineered human primary T cells as described above in FIG. 5 were cultured until day 10. Following, the engineered human primary T cells were reactivated by an activator of TCR (e.g., OKT3/CD28) for 3 days. Subsequently, PD1 expression level in the in silico sorted populations of Zap70+ (a combination of Ldck+Zap70+ and Zap70+ only), Ldck+Zap70+, Zap70+ only, Ldck+ only, or double negative (−/−) human primary T cells was measured (e.g., by flow cytometry) and plotted, to compare the differences between NOsg and PD1sg conditions. Upon the reactivation of the cells, the engineered human primary T cells comprising both Ldck construct and Zap70 construct exhibited the greatest decrease (e.g., about 38% decrease) in the expression level of PD1 (or the greatest decrease in the proportion of the cells expressing PD1) between NOsg and PD1sg conditions, as compared to other control cells.

### Example 3: Formation of a Signaling Complex of an Endogenous Receptor by a First Chimeric Polypeptide and a Second Chimeric Polypeptide, Using γ-Retrovirus

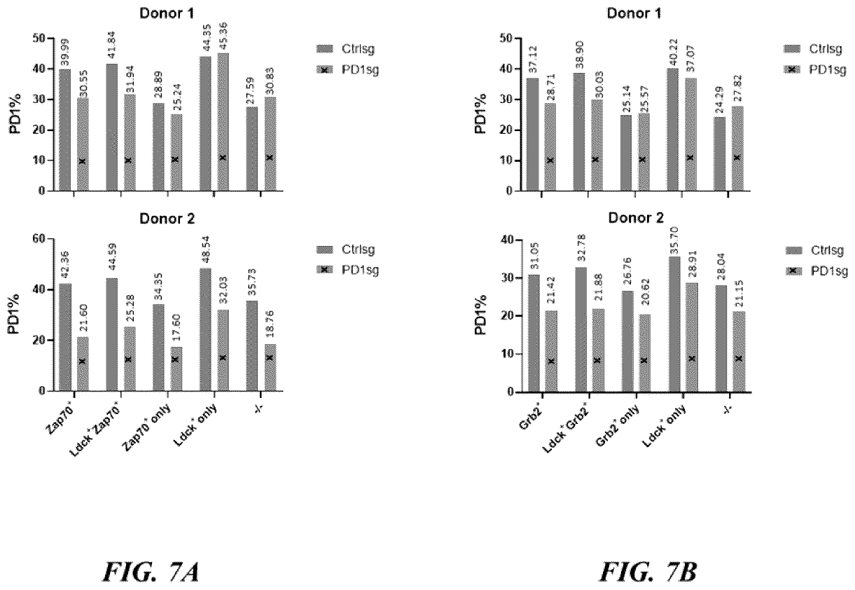

Human primary T cells (Donor 1 or Donor 2, as indicated) were activated by a TCR activator (e.g., OKT3/CD28) at day 0, followed by transduction of one or more γ-retroviruses, each virus encoding LAT.dCas9KRAB.Q8 (“Ldck”), Zap70.TEV.tNGFR/PD1sg, Zap70.TEV.tNGFR/Ctrlsg, Grb2.TEV.tNGFR/PD1sg, or Grb2.TEV.tNGFR/Ctrlsg. PD1sg denotes a sgRNA against PD1 gene. Ctrlsg denotes a control sgRNA that is not designed to target PD1 gene. At day 6, a double positive population of Q8+tNGFR+ human primary T cells was isolated and enriched via cell sorter. The sorted cells were further expanded until day 14, followed by T cell reactivation by an actuator or TCR (e.g., OKT3/CD28) for 3 days. Subsequently, PD1 expressions in the cells were measured and plotted to compare the differences between NOsg and PD1sg conditions (FIGS. 7A and 7B).

Referring to FIG. 7A, following the expansion and reactivation of the Q8+tNGFR+ double positive human primary T cells from Donor 1, the cells were further sorted in silico into the following groups: Zap70+ (a combination of Ldck+Zap70+ and Zap70+ only), Ldck+Zap70+, Zap70+ only, Ldck+ only, or double negative (−/−) human primary T cells, then a proportion of PD1 positive cells in each group was plotted, thereby to compare the differences between NOsg and PD1sg conditions. Upon the expansion and reactivation of the cells, the engineered human primary T cells exhibiting the highest expression level of both Ldck construct and Zap70 construct (“Ldck+Zap70+”) exhibited the greatest decrease (e.g., about 23% decrease) in the expression level of PD1 (or the greatest decrease in the proportion of the cells expressing PD1) between NOsg and PD1sg conditions, as compared to other control cells. For example, upon the expansion and reactivation of the cells, the engineered human primary T cells exhibiting a similar expression level of Ldck construct and a lower expression level of Zap70 construct (“Ldck+ only”) did not exhibit any decrease in the expression level of PD1 (or that of the proportion of the cells expressing PD1) between NOsg and PD1sg conditions. In another example, upon the expansion and reactivation of the cells, the engineered human primary T cells exhibiting a similar expression level of Zap70 construct and a lower expression level of Ldck construct (“Zap+ only”) exhibited only about 12% decrease in the expression level of PD1 (or that of the proportion of the cells expressing PD1) between NOsg and PD1sg conditions.

Referring to FIG. 7A, for Donor 2, all groups that were sorted in silico exhibited a decrease in the expression level of PD1 (or that of the proportion of the cells expressing PD1) between NOsg and PD1sg conditions. All of the cells were from double positive population of Q8+tNGFR+ human primary T cells. Thus, without wishing to be bound by theory, even though the cells were sorted in silico into different groups depending on their relative expression levels of Zap70 and Ldck constructs, it may be possible that the cells in all of the groups expressed sufficient levels of both Zap70 and Ldck constructs to elicit the decrease in the expression of PD1 in the presence of PD1sg.

Referring to FIG. 7B, following the expansion and reactivation of the Q8+tNGFR+ double positive human primary T cells from Donor 1, the cells were further sorted in silico into the following groups: Grb2+ (a combination of Ldck+Grb2+ and Grb2+ only), Ldck+Grb2+, Grb2+ only, Ldck+only, or double negative (−/−) human primary T cells, then a proportion of PD1 positive cells in each group was plotted, thereby to compare the differences between NOsg and PD1sg conditions. Upon the expansion and reactivation of the cells, the engineered human primary T cells exhibiting the highest expression level of both Ldck construct and Grb2 construct (“Ldck+Grb2+”) exhibited the greatest decrease (e.g., about 22% decrease) in the expression level of PD1 (or the greatest decrease in the proportion of the cells expressing PD1) between NOsg and PD1sg conditions, as compared to other control cells. For example, upon the expansion and reactivation of the cells, the engineered human primary T cells exhibiting a similar expression level of Grb2 construct and a lower expression level of Ldck construct (“Grb2+ only”) did not exhibit any decrease in the expression level of PD1 (or that of the proportion of the cells expressing PD1) between NOsg and PD1sg conditions. In another example, upon the expansion and reactivation of the cells, the engineered human primary T cells exhibiting a similar expression level of Ldck construct and a lower expression level of Grb2 construct (“Ldck+ only”) exhibited only about 7% decrease in the expression level of PD1 (or that of the proportion of the cells expressing PD1) between NOsg and PD1sg conditions.

Referring to FIG. 7B, for Donor 2, all groups that were sorted in silico exhibited a decrease in the expression level of PD1 (or that of the proportion of the cells expressing PD1) between NOsg and PD1sg conditions. All of the cells were from double positive population of Q8+tNGFR+ human primary T cells. Thus, without wishing to be bound by theory, even though the cells were sorted in silico into different groups depending on their relative expression levels of Grb2 and Ldck constructs, it may be possible that the cells in all of the groups expressed sufficient levels of both Grb2 and Ldck constructs to elicit the decrease in the expression of PD1 in the presence of PD1sg.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of regulating expression of a target polynucleotide in a cell, comprising:
(a) expressing a system in the cell, wherein the cell comprises a receptor having a ligand binding domain specific for a ligand; and
(b) contacting the cell with the ligand that binds specifically the ligand binding domain, wherein the system expressed in the cell comprises:
a first chimeric polypeptide and a second chimeric polypeptide that are activatable upon the contacting step (b), wherein one of the first and second chimeric polypeptides comprises a gene modulating polypeptide (GMP) comprising an actuator moiety linked to a cleavage recognition site, which actuator moiety is capable of regulating the expression of the target polynucleotide in the cell, and wherein the other of the first and second chimeric polypeptides comprises a cleavage moiety capable of cleaving the cleavage recognition site of the GMP,
wherein, upon the contacting of the cell by the ligand that binds specifically the ligand binding domain of the receptor, the first and second chimeric polypeptides are activated such that the cleavage moiety cleaves the cleavage recognition site and releases the actuator moiety from the GMP, thereby regulating the expression of the target polynucleotide in the cell, and
wherein the receptor is an endogenous receptor of the cell.

2. The method of claim 1, wherein the first chimeric polypeptide comprises a first adaptor moiety that is activatable to bind a first intracellular domain of the endogenous receptor, and
wherein the second chimeric polypeptide comprises a second adaptor moiety that is activatable to bind (i) a second intracellular domain of the endogenous receptor, (ii) the first adaptor moiety, or (ii) a downstream signaling moiety of the endogenous receptor.

3. (canceled)

4. The method of claim 1, wherein the first chimeric polypeptide comprises a first adaptor moiety that is activatable to bind a first downstream signaling moiety of the endogenous receptor, and
wherein the second chimeric polypeptide comprises a second adaptor moiety that is activatable to bind (i) the first adaptor moiety, (ii) the first downstream signaling moiety, or (iii) a second downstream signaling moiety of the endogenous receptor.

5-13. (canceled)

14. The method of claim 1, wherein the first chimeric polypeptide comprises the GMP, and wherein the second chimeric polypeptide comprises the cleavage moiety, or
wherein the second chimeric polypeptide comprises the GMP, and wherein the first chimeric polypeptide comprises the cleavage moiety.

15. (canceled)

16. The method of claim 1, wherein the first and second chimeric polypeptides are activatable upon the contacting step (b) to form a signaling complex of the receptor.

17-26. (canceled)

27. A system for regulating expression of a target polynucleotide in a cell, comprising:
a first chimeric polypeptide and a second chimeric polypeptide, wherein one of the first and second chimeric polypeptides comprises a gene modulating polypeptide (GMP) comprising an actuator moiety linked to a cleavage recognition site, which actuator moiety is capable of regulating the expression of the target polynucleotide in the cell, and wherein the other of the first and second chimeric polypeptides comprises a cleavage moiety capable of cleaving the cleavage recognition site of the GMP,
wherein the cell comprises a receptor having a ligand binding domain specific for a ligand, wherein the first and second chimeric polypeptides are activatable upon contacting of the cell by the ligand that binds specifically the ligand binding domain of the endogenous receptor,
wherein, upon the contacting of the cell by the ligand, the first and second chimeric polypeptides are activated such that the cleavage moiety cleaves the cleavage recognition site and releases the actuator moiety from the GMP, thereby regulating the expression of the target polynucleotide in the cell,
wherein the receptor is an endogenous receptor of the cell.

28. The system of claim 27, wherein the first chimeric polypeptide comprises a first adaptor moiety that is activatable to bind a first intracellular domain of the endogenous receptor.

29. The system of claim 28, wherein the second chimeric polypeptide comprises a second adaptor moiety that is activatable to bind (i) a second intracellular domain of the endogenous receptor, (ii) the first adaptor moiety, or (ii) a downstream signaling moiety of the endogenous receptor.

30. The system of claim 27, wherein the first chimeric polypeptide comprises a first adaptor moiety that is activatable to bind a first downstream signaling moiety of the endogenous receptor.

31. The system of claim 30, wherein the second chimeric polypeptide comprises a second adaptor moiety that is activatable to bind (i) the first adaptor moiety, (ii) the first downstream signaling moiety, or (iii) a second downstream signaling moiety of the endogenous receptor.

32-39. (canceled)

40. The system of claim 27, wherein the first chimeric polypeptide comprises the GMP, and wherein the second chimeric polypeptide comprises the cleavage moiety.

41. The system of claim 27, wherein the second chimeric polypeptide comprises the GMP, and wherein the first chimeric polypeptide comprises the cleavage moiety.

42. The system of claim 27, wherein the first and second chimeric polypeptides are activatable upon the contacting to form a signaling complex of the receptor.

43-44. (canceled)

45. The system of claim 27, wherein the receptor comprises at least a portion of T cell receptor (TCR), and
wherein the TCR comprises a co-receptor of TCR, comprising CD3, CD4, or CD8.

46. (canceled)

47. The system of claim 27, wherein an intracellular domain the receptor comprises at least one immunoreceptor tyrosine-based activation motif (ITAM).

48. The system of claim 28, wherein the first adaptor moiety or the second adaptor moiety comprises LCK, FYN, ZAP-70, LAT, SLP76, ITK, PLC-γ, VAV1, NCK, GADS, GRB2, PI3K, a fragment thereof, or a combination thereof.

49. The system of claim 27, wherein the receptor comprises at least a portion of NKG2D.

50. The system of claim 47, wherein the first adaptor moiety or the second adaptor moiety comprises DAP10, DAP12, PI3K, GRB2, VAV1, SYK, ZAP-70, a fragment thereof, or a combination thereof.

51. The system of claim 27, wherein the receptor comprises at least a portion of Toll-like receptor (TLR) selected from the group consisting of: TLR1, TLR2, TLR3, TLR4, TLR5, TLR6, TLR7, TLR8, TLR9, TLR10, TLR11, TLR12, and TLR13.

52. The system of claim 50, wherein the first adaptor moiety or the second adaptor moiety comprises MyD88, Tube, Pelle, TIRAP, TRIF, TRAM, IRAK1, TRAK4, TRAF6, TAK1, TBK1, RIPK1, PI3K, IKK, a fragment thereof, or a combination thereof.
